(19) United States
(12) Patent Application Publication
(10) Pub. No.: US 2025/0064793 A1
(43) Pub. Date: Feb. 27, 2025

(54) EDIBLE ORAL FILMS, COMPOSITIONS, METHOD OF MANUFACTURE AND PACKAGING SYSTEMS

(71) Applicants: Madhu Hanharan, Greensboro, NC (US); Joseph Fuisz, Nashville, TN (US)

(72) Inventors: Madhu Hanharan, Greensboro, NC (US); Joseph Fuisz, Nashville, TN (US)

(21) Appl. No.: 18/629,816

(22) Filed: Apr. 8, 2024

Related U.S. Application Data
(60) Provisional application No. 63/611,648, filed on Dec. 18, 2023.
(60) Provisional application No. 63/542,495, filed on Oct. 4, 2023.
(60) Provisional application No. 63/534,574, filed on Aug. 24, 2023.

(51) Int. Cl. A61K 31/465; A61J 1/14; A61K 9/00; A61K 31/00; A61K 47/02; A61K 47/14; A61K 47/18; A61K 47/26; A61K 47/34; A61K 47/38; A61K 47/46

(52) U.S. Cl. CPC A61K 31/465; A61J 1/14; A61K 9/006; A61K 31/658; A61K 47/02; A61K 47/14; A61K 47/183; A61K 47/26; A61K 47/34; A61K 47/38; A61K 47/46

(57) ABSTRACT

Embodiments include a film or sheet manufactured by dynamic roll molding. The film or sheet can include an active agent (e.g., nicotine) for oral administration. In aspects, an active agent is applied to the oral mucosa of a subject for local or systemic distribution. Embodiments also include containers and dispensers for orally soluble and edible films or sheets for administering therapeutic agents. The film or sheet can include an active agent (e.g., nicotine) for oral administration. In aspects, an active agent is applied to the oral mucosa of a subject for local or systemic distribution. Embodiments also include containers that dispense orally soluble and edible films or sheets via rotational cassettes which align film or sheet-containing chambers with an open aperture.

### RELATED APPLICATIONS

This application claims priority to U.S. Patent Application No. 63/534,574 filed Aug. 24, 2023, U.S. Patent Application No. 63/542,495 filed Oct. 4, 2023, and U.S. Patent Application No. 63/611,648 filed Dec. 18, 2023. The contents of the aforementioned applications are herein incorporated by reference.

### TECHNICAL FIELD

The present disclosure relates generally to delivery of therapeutic agents, and more specifically, to edible oral soluble films for administering pharmacologically active agents.

### BACKGROUND

As their name suggests, an “oral thin film” (OTF) or “oral soluble film” is a thin film composed of a mono or multi-layer polymeric matrix that is applied in the mouth. They can be water-based soluble polymers designed to rapidly deliver drugs systemically. Depending on the drug candidate and the targeted indication, they can be formulated either as orodispersible films or a transmucosal film. Orodispersible films (ODF's) work like conventional orodispersible tablets. An ODF typically disintegrates or dissolves in seconds in the mouth and the drug substance is swallowed. With a transmucosal film, the active substance directly enters the systemic circulation via the buccal mucosa. The latter approach allows a medication to bypass the first pass metabolism thereby making the medication more bioavailable.

Thin-film drug delivery uses a dissolving film or oral drug strip to administer drugs via absorption in the mouth (i.e., buccally or sublingually) and/or via the small intestines (i.e., enterically). A film is prepared using hydrophilic polymers that rapidly dissolves on the tongue or buccal cavity, delivering the drug to the systemic circulation via dissolution when contact with liquid is made.

Thin-film drug delivery has emerged as an alternative to the traditional tablets, capsules and liquids often associated with prescription and OTC medications. Similar in size, shape and thickness to a postage stamp, thin-film strips are typically designed for oral administration. A user places the strip on or under the tongue (i.e., sublingual) or along the inside of the cheek (i.e., buccal). As the strip dissolves, the drug can enter the blood stream enterically, buccally or sublingually. For systemic transmucosal drug delivery, the buccal mucosa is generally preferred compared to the sublingual mucosa.

To date, “wet casting” or solvent casting of films has been the dominant method of manufacture of commercially available oral soluble films. Solvent casting is discussed in U.S. Pat. No. 7,897,080 which describes a method of making films by deposition as an alternative to the solvent casting process. In contrast, “hot melt extrusion” to manufacture edible, soluble films and sheets is described in the art. However, commercially, the hot melt extrusion process (and underlying extrudable compositions suitable for manufacture by hot melt extrusion) has not yielded commercial products to the knowledge of the Applicants. Rather, commercially sold oral soluble films are made by the wet or solvent casting process.

Dispensers for edible oral films are known in the art. The most paradigmatic example being Listerine PocketPaks®, an edible oral film and edible oral film dispenser that was first launched in October 2001. Listerine PocketPaks® are small, rectangular, single-chambered cassettes that hold a stack of multiple orally soluble films in a compartment that is accessible by a flip-top lid on the front of the cassette. The cassettes are stored in a secondary blister package composed of Aclar® film which provides barrier protection to the orally soluble films until the cassette is removed from the secondary blister package. The orally soluble films use pullulan as the primary film former. However, pullulan has severe loading limitations and so the behavior and attributes of primarily-pullulan based films is sui generis. The film-facing interior wall of the cassette is smooth-textured to allow easy sliding of the orally soluble films from the cassette without blockage or adhesion.

Various companies have manufactured products with similar packaging, as unit dose sachets, wherein the film is packed as a unit dose, typically in a foil-foil laminate. Such products include approved ethical drugs sold in soluble films; over-the-counter (OTC) drug products; nutraceutical products as well as films containing cannabinoids. Examples of oral soluble film ethical drugs include Suboxone® (unit dose pouch), Sympazam® (unit dose pouch), Igalmi® (unit dose pouch), Kynmobi® (unit dose pouch), Zuplenz® (unit dose pouch), etc. OTC drug products include oral soluble films previously marketed under Triaminic® (unit dose pouch), Benadryl® (unit dose pouch) and Gas-x® (unit dose pouch). Nutraceutical oral films include Sanofi's Unisom® dissolving strips (multi-dose cassette). Cannabinoid containing films include KinSlips® (unit dose pouch).

Schiraldi et al. (U.S. RE33,093) describes bioadhesive monolayer extruded films, under 10 millimeters in thickness (10 thousandths of an inch), composed principally of polyethylene oxide together with HPC, a water insoluble polymer; a plasticizer and a medicament. Similarly, U.S. Pat. No. 6,072,100 describes compositions extruded films and sheets comprising PEO or HPC, a water polymer derived from a carboxylic aid, 30-80% plasticizer and up to 10% of a medicament.

Michael Repka and James McGinnity disclose hot melt extruded sheets with a thickness of 10-13 millimeters using a 50:50 ratio PEO and HPC, together with 3% of Vitamin E TPGS (see “Influence of Vitamin E TPGS on the properties of hydrophilic films produced by hot melt extrusion,” International Journal of Pharmaceutics 202 (2000) 63-70).

U.S. Pat. No. 6,375,963 disclose a hot-melt extruded film and method of preparation. The inventors note that “[f]ilms comprising pure hydroxypropylcellulose (HPC) and other water-soluble or water-swellable polymers cannot be readily produced by hot-melt extrusion due to the high stress that is exhibited on the extruder. Therefore plasticizers have been added to the HPC and other polymers” and that “the prior art does not disclose that films comprising a major portion of HPC and other water-soluble or water-swellable polymers can be produced by hot-melt extrusion in the absence of a plasticizer.” To solve this problem Repka et al propose using a bioadhesive polymer instead of a plasticizer. The film is made from a precursor composition containing a water soluble or water swellable thermoplastic polymer (e.g., HPC and/or PEO) and a bioadhesive polymer. The film does not contain a conventional plasticizer or a material that is recognized as a plasticizer. Repka et al describe films in their Examples comprised of up to 10% active agent.

U.S. Patent Publication 2006/0257463 describes transmucosal film compositions for delivery of tetrahydrocannabinol (THC). Examples include hot melt molding, solvent casting and hot melt extrusion. Hot melt extrusion examples comprise PEO and 8% w/w THC or 8% w/w THC-HS. The preparation of Examples 5 & 6 are described as follows: “Example #5 and #6 were prepared using hot-melt extrusion techniques. The PEO, PVP and Vitamin E TPGS were blended in a V-blender. The THC and the THC-HS were solubilized in the PEG 400 and immediately sprayed into the dry blend with continuous mixing. The resulting blend was then hot-melt extruded into films. The highest extrusion temperature was 150 C and residence time in the barrel was approximately 2 minutes. The resulting transmucosal preparations were approximately 1.0 mm in thickness and both contained over 98% of the original theoretical percent of drug within the formulation.”

Elsohly does not provide a formal description of “hot melt molding,” but he does provide a description of that process in the above referenced patent application: “Example #1 was prepared by hot-melt molding. The Tetrahydrocannabinol (THC) was dissolved within the Polyetheylene glycol 400. The other Inner matrix components were then mixed and heated to approximately 140° C. and homogeneously blended. The solubilized THC was then slowly added to the heated admixture and dispersed. The resulting molten matrix was then poured into a film mold to obtain a uniformly thick (approximately 1.5 mm) film after slowly cooling. The backing layer was adhered with 40° C. heating. The Outer backing ingredients were heated (90° C.), mixed and molded separately.” Ingredients for the inner layer of this Eloshly Example 1 include: Klucel (EF grade), Polycarbophil, PEG 400, tartaric acid, BT and TJC. The “outer backing layer” comprises Klucel (EF Grade), Ethyl Cellulose, Eudragit and PEG 3350.

U.S. Pat. No. 10,334,872 issued Jul. 2, 2019, describes extruded sheets containing tobacco manufactured by hot melt extrusion at temperatures. The '872 patent provides examples of hot melt extruded sheets containing up to 30% tobacco (Bruton Snuff) (See Example G describing an extruded hot melt sheet of 13 mils. The '872 patent Example O describes a clinical study with a sheet containing 75 mg of tobacco, with an implied sheet piece weight of 300 mg.

U.S. Pat. No. 8,613,285 issued Dec. 24, 2013, describes extruded sheets containing bioactive agents. The '285 patent further describes the use of a drug in an ion exchange in an extrudable composition, and the ability of the ion exchange complex to remain bonded through the hot melt extrusion process. Elsohly US 2006/0257463 discloses transmucosal film compositions for the delivery of tetrahydrocannabinol (THC).

As alluded to above, pharmaceutically and commercially available oral thin film products have typically employed unit-dose foil-foil sachets. In these typical constructions, each foil-foil sachet contains a single film, and often includes a child resistance feature which requires a special tearing sequence to ready the dosage of oral film for use. Further, the films rapidly dissolve and can hold a small amount of an active agent or medicament. Accordingly, there exists an unmet need for orally soluble film that dissolves slowly and can hold higher loads of agent. There is also a need for dispensers to administer orally soluble films and sheets in an efficient manner without the added cost and inconvenience (including without limitation trash burden) of foil packaging for individual dosages while maintaining separation of each individual film unit.

### SUMMARY OF INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims, drawings, and abstract as a whole.

Historically, oral thin film products are either square shaped or rectangular with a length by width aspect ratio ranging from 1:1 to 1.5:1. For oral thin films with dimensions greater than 1.5:1 up to 4:1 which are more suitable for buccal use, there exists a need for a dispensing container containing multiple films physically isolated from one another but allowing for dispensing of one film at a time. It is an object of certain embodiments of the present invention to provide a unit dose container for films and sheets of such aspect ratios, including a unit dose container suitable for the consumer space suitable for placement in the pocket of a user.

It is desirable for certain products—particularly consumer products—to avoid the need for foil-foil sachet packaging of individual strips. This is for reasons of cost and convenience by obviating the need to open and dispose of an individual sachet for each film or sheet dispensed. Litter and related environmental issues can similarly be mitigated.

It is an object of the present invention to provide for a film or sheet that wherein multiple said films or sheets may be packed loosely in a container without individual film/sheet packages. It is a further object of the present invention that films or sheets do not adhere together when stored at ambient conditions. In certain embodiments, the films or sheets are not tacky when packaged together in a container with loose films subjected to 30 days of 25 C/60% relative humidity (RH).

It is an object of the present invention to provide an orally soluble film and sheet dispenser that includes individual chambers for each individual film or sheet such that the films or sheets are not packaged in individual sachets and are not in contact with other films or sheets.

It is another object of the invention to provide a round cassette container with multiple chambers and rotational capability, such that the cassette can be rotated to stop at various angular positions to align a dispensable product with an aperture or opening on the cassette which allows for the removal of the film or sheet contained in the cassette, including one film or sheet at a time from a multi film/sheet container.

It is a further object of the invention to provide a round cassette container with a single chamber and rotational capability, such that the cassette can be rotated to stop at various angular positions to align a dispensable product with an aperture or opening on the cassette which allows for the removal of the film or sheet contained in the cassette. A round cassette is particularly suitable for longer and thinner films with length by width aspect ratios greater than 1.5 to 1

It is a further object of the invention to provide for a film or sheet that is rigid such that it resists bending or curling and remains substantially flat within the cassette chamber allowing easy removal through an aperture.

It is an object of the invention to provide for a film or sheet manufactured by dynamic roll molding. In aspects, the film or sheet includes a form of nicotine, and/or a cannabinoid.

It is another object of the invention to provide a round multi-chambered cassette container with rotational or other movement capability. In aspects, each chamber of the cassette can move within the cassette and stop in alignment with an aperture or opening on the cassette. This allows for removal of a film or sheet contained in each chamber.

In embodiments, the cassette has a single chamber with multiple films or sheets arranged sequentially within the chamber using clips, brushes or pins to hold the films or sheets in place.

In embodiments, the cassette includes a desiccant. The desiccant may be a single desiccant pack (or area) or multiple packs or areas. Preferably, the desiccant is not in direct, physical contact with the films or sheets. In some embodiments, the desiccant is housed in an area of the cassette which has one or more air channels to allow for the transfer of moisture from the film/sheet area to the desiccant area or areas.

In some embodiments, the cassette is packaged with an overwrap (i.e. a packaging film over the cassette). The overwrap can provide a moisture barrier to ensure stability of the films or sheets. While many types of material are suitable, a preferred overwrap material is polypropylene, including biaxially-oriented polypropylene (BOPP). The cassette can be entirely or partially overwrapped.

In some embodiments, the “joints” or “seams” of the cassette are covered. In aspects, a suitable moisture barrier film is employed.

In other embodiments, the cassette has multiple chambers with multiple films or sheets arranged sequentially within the chamber using clips, brushes, pegs or pins to hold the films or sheets in place. In further aspects, the cassette is a round container with rotational or other movement capability such that no chamber can move until a film or sheet is removed from the chamber aligned with the aperture.

In other embodiments of the invention, a container or cassette cover has rotational capability, such that the container or cassette cover can rotate to align with an aperture in the cover and stop at a given chamber to allow for the removal of a film or sheet contained in the corresponding chamber aligned with the aperture.

In certain embodiments, rotation of the cassette, chamber, opening, or aperture is effected through the use of a dial. In other embodiments, rotation of the cassette, chamber, opening or aperture is achieved with a push button actuator that creates rotation through a gear. Comparable and equivalent mechanisms known in the art may also be employed to effect rotation of the cassette, chamber, opening or aperture of the present invention. Other actuators that are also contemplated, include a push-button actuator, a piezoelectric actuator, a Bluetooth actuator, a fingerprint actuator, a switch actuator, a spring-loaded actuator or a dial.

In other embodiments, the container includes a rectangular cassette which slides linearly within a larger rectangular case or container cover which has an aperture. The cassette may be pushed along the case to align each chamber with the aperture to allow removal of individual films within each chamber of the cassette.

In additional embodiments, the invention includes a container cover with rotational capability, such that the container cover is capable of rotating to align an aperture in the cover to stop at a given chamber to allow for the removal of a film or sheet contained in the corresponding chamber.

In further aspects, the invention contemplates a container capable of dispensing individual films or sheets to a user by gravity. More specifically, the individual films or sheets may be dispensed by dropping from the container through an aperture due to the force of gravity when used as intended. In other embodiments, tapping or bumping of the container against a surface may be required to shake loose a film via an aperture.

It is an object of the invention to provide for a film or sheet manufactured by dynamic roll molding, further comprising at least one active pharmaceutical ingredient.

It is an object of the invention to provide for a film or sheet manufactured by dynamic roll molding, comprising a breath freshener or mint containing menthol, eugenol, peppermint oil, eucalyptol, thyme, fennel or other breath freshening agents.

It is an object of the invention to provide for a film or sheet manufactured by dynamic roll molding, wherein said film dissolves, when placed in the buccal cavity, in five to seventy minutes, preferably in ten to forty-five minutes, more preferably in fifteen to thirty minutes.

It is an object of the invention to provide for a film or sheet manufactured by dynamic roll molding, wherein the final film or sheet product has an average thickness of 1 to 50 mils, preferably 8 to 20 mils, most preferably 9 to 12 mils.

It is an object of the invention to provide for a film or sheet manufactured by dynamic roll molding, wherein said film has a textured surface to increase wettability and disintegration in the lingual cavity.

In yet other aspects, the invention includes a container for dispensing individual films or sheets to a user by mechanical force applied to an individual film or sheet to raise, present, or eject the individual film or sheet from the container.

In certain embodiments, containers of the invention are assembled from four pieces of fewer, preferably three pieces or fewer, more preferably two pieces or fewer (in each case not including overwrap). In embodiments, such pieces are injection molded.

In other embodiments, the films are loosely packaged within a tin or puck type package made of polymeric or other resin or metal which may or may not be airtight. These tins or pucks can contain a cone or reverse cone shaped insert to allow easy sliding removal of the films within the package. The top of the cone may reach the height of the interior space. Preferably, the top of the cone reaches at least two-thirds of the height of the interior space, more preferably at least three fourths, most preferably at least four fifths.

In yet other embodiments, the films are arranged within slots on the cone-shaped insert which can be arranged radially like spokes to allow easy removal of a single film by pinching a film between two fingers and sliding or lifting. Other non-radial slot configurations are similarly contemplated. The insert can be a separately made material that is placed in the container, or the insert can be manufactured as part of the container or puck.

In other embodiments, the invention includes a non-smooth surface within a dispensing container. The non-smooth surface provides a non-smooth texture to retard the sticking of the films or sheets to the non-smooth surface. For example, a non-smooth surface resembling a grate may be employed to reduce the contacting surface area exposed to the films or sheets. Other surface textures contemplated include a pocked texture, a rough texture, a knurled texture, an uneven texture, a studded texture, a combination of any of the aforementioned and any equivalent texture or texture combinations that are known in the art.

In other embodiments, the inside surface of the dispensing container is coated with a hydrophobic material. The hydrophobic coating can be silicone, or any other oily hydrophobic substance. The hydrophobic material can be admixed with the polymeric material of construction of the dispensing container. The container may be opaque, translucent or transparent.

It is a further object of the invention to provide a chamber for a film or sheet such that the contact surface area of the container is mostly composed of a low-surface-energy substrate (i.e., a substrate with a surface energy of 40 dynes/cm or lower, preferably 30 dynes/cm or lower). In certain embodiments, a low-surface-energy substrate is paired with a rough or pocked surface texture. The substrate and surface texture can be parallel to each other, orthogonal, opposing, adjacent, or in any configuration within the grasp of a skilled artisan.

The textures, surface energy and other attributes discussed above may equally apply to a puck style container.

In yet other aspects, the bottom of the chamber is sloped upwards. The bottom of the chamber may further include a single upward planar surface. The bottom of the chamber may include two or more upward planar surfaces. The planar surfaces may include a constant slope, but concave, convex and other curved surfaces are also contemplated.

Low surface energy contact areas and rough or pocked textures are particularly suitable for use with films or sheets that include oily active ingredients. Examples include nicotine oil, or cannabinoids. Product tackiness for a film or sheet may increase as the film matrix comprises more than 3% of an oily ingredient (e.g. nicotine oil). Such tackiness typically increases with increased oil percentages (i.e. 5% or greater, and still more tackiness at 7.5% or greater). Cannabinoids show similar properties; increased product tack above the prior percentages. Nicotine and cannabinoid containing films and sheets at these loading levels are expressly contemplated.

In certain embodiments, “dusting” is employed to reduce tackiness where films or sheets to reduce product tackiness (i.e. placing a powder in the container, optionally hydrophobic, that reduces tackiness of the films or sheets one with the other). Without limitation, corn starch may be suitable for dusting.

In certain embodiments, a chamber of the invention contains multiple films or sheets. In further embodiments, the chamber is sized to encourage the movement of multiple films or sheets.

In other embodiments, the chamber size is such that there is minimization of contact of the flat surface of the film with the surfaces of the chamber. In further embodiments, the chamber is rectangular and has sides with a smaller width than the film or sheet so that the film or sheets can only be placed along the diagonal width of the chamber, ensuring that only the edges of the film or sheet make contact with the corners of the chamber.

Is aspects, the container employs gaskets to reduce air and vapor flux. Ideally, the container is airtight, or substantially airtight. Whether the container is airtight can be considered both at the point when the container has an overwrap, as well as after the overwrap has been removed.

Child resistance features may be incorporated in the lid of the container or aperture or by any other suitable means such as pin-actuated enablement of cassette rotation, or pressure actuated enablement. Such features may apply to all containers described or contemplated herein.

In other embodiments, the aperture of the container is configured to push or pull out the individual film or sheet from the chamber, including through physical contact (i.e., grasping and pulling or pushing) with the film or sheet.

In other embodiments, the cassette/container of the invention includes nylon brush hairs within the chambers. Each chamber can include protruding brush hairs such that each film or sheet may be emplaced within the brush hair fibers in a radial arrangement to hold the film or sheets firmly in place while ensuring minimal contact of the film or sheet with brush hairs while also allowing removal of film or sheet embedded in the brush hair when presented at the aperture. The fibers can protrude from or along any of the walls of the chamber. The fibers can also be parallel to the plane of the film or sheet or perpendicular to the plane of the film or sheet or at any angle there between.

In further embodiments, the container/cassette includes brush fibers that can be used to hold each film or sheet in lieu of the use of chambers. Each film or sheet can be embedded within the brush hairs of the rotating or sliding cassettes/containers of the invention.

It is an object of the present invention to provide for a film or sheet for buccal or sublingual use.

It is an object of the present invention to provide for a film or sheet with a textured surface for maximizing oral absorption and preventing tackiness.

It is an object of the present invention to provide for a film or sheet that is color-coded to flavor, i.e., the color of the film or sheet matches the flavor (e.g., orange for orange).

It is an object of the present invention to provide for a film or sheet comprising meglumine as a pH modifier. Optionally, said film or sheet further comprises an active ingredient, including without limitation nicotine, a salt thereof, a complex thereof, or a similar molecule.

It is an object of the present invention to provide for a film or sheet that includes meglumine as an absorption enhancer for basic drugs.

It is an object of the present invention to provide for a film or sheet that includes arginine as an absorption enhancer for basic drugs.

It is an object of the present invention to provide for a film or sheet that includes glyceryl monostearate as an absorption enhancer.

It is an object of the present invention to provide a film or sheet that includes polyoxyl-32 stearate as an absorption enhancer.

It is an object of the present invention to provide a film or sheet that includes triglyceryl monooleate as an absorption enhancer.

It is an object of the present invention to provide for a film or sheet that includes sodium caprate as an absorption enhancer.

It is an object of the present invention to provide for a film or sheet that includes meglumine to reduce process temperature.

It is an object of the present invention to provide for a manufacturing process and compositions suitable for manufacturing a film or sheet, wherein processing temperatures do not exceed 120° C., preferably do not exceed 110° C., more preferably do not exceed 100° C., and most preferably do not exceed 90° C.

It is an object of the present invention to provide for a manufacturing process and compositions suitable for manufacturing a film or sheet, wherein processing temperatures do not exceed 120° C., preferably do not exceed 110° C., more preferably do not exceed 100° C., and most preferably do not exceed 90° C.; where the film or sheet comprises less than 20% active ingredient, preferably less than 15% active ingredient, more preferably less than 10% active ingredient, most preferably less than 5% active ingredient.

It is an object of the present invention to provide for a manufacturing process and compositions suitable for manufacturing a film or sheet, wherein processing temperatures do not exceed 120° C., preferably do not exceed 110° C., more preferably do not exceed 100° C., and most preferably do not exceed 90° C.; where the film or sheet comprises 75% or more of the dry composition weight of the feedstock, preferably 80% of more of the dry composition weight of the feedstock, most preferably more than 85% of the dry composition weight of the feedstock.

It is an object of the present invention to provide for compositions and process suitable for manufacturing a film or sheet, wherein the certain ingredients are melted and certain ingredients remain unmelted during the manufacturing process. Preferably, molten inactive components comprise 40% to 90% of the composition, more preferably 50%-80% and most preferably 60-70% of the composition.

It is an object of the present invention to provide for a film or sheet comprising nicotine with a pH of greater than 7, preferably greater than 8, and most preferably greater than 9. In preferred embodiments, said nicotine film does not comprise sodium carbonate or sodium bicarbonate, or other similar basic salts. It is an object of the present invention to provide a film or sheet that is sufficiently non-hygroscopic, to allow for stable bulk packaging in a container. For example, 10-40 films or sheets may be placed loosely in a container.

It is an object of the present invention to provide for a film or sheet with adequate stiffness to be easily placed by a user into the buccal cavity without requiring folding to accommodate within the buccal cavity. Film stiffness as described here is similar to bending strength and flexural strength in engineering material characterization. The American Society of Testing and Materials (ASTM) methods ASTM D2344 and D790 are two examples of standards used to calculate these types of properties. The stiffness of the film was quantified using an apparatus similar to that described for a 3-point test in the ASTM standards. Here, films of 0.4 mm thickness, 11 mm wide and 33 mm long were considered as a beam and tested on a 3-point testing apparatus wherein the film was supported on two points at the end and the force in milliNewtons (mN) was measured as a third point crosshead probe pushed at the center of the film to deflect it by 1 mm at 1.1 mm/min speed. The average force required to bend the film is 100-600 mN, more preferably 200-500 mN and most preferably 300-400 mN. The peak force required to bend a film of 0.3 mm thickness is 50-400 mN, more preferably 75-300 mN and most preferably 100-200 mN. These values indicate that these films have higher stiffness than typical oral films as found in the art which may have stiffness values only ranging from a 2 mN to 25 mN.

It should be noted that in embodiments where the dynamic roll molding process results in a web wherein pieces are cut in line, the products may have reduced bendability as compared with typical cast films, where the film web is rolled up after drying prior to being cut into pieces.

It is an object of the invention to provide for a film or sheet for buccal use wherein the film has dimensions (measuring from the longest and tallest points) of 1.5-5 (length) to 1 (height); preferably 2.0-3.25 to 1, more preferably 2.5-3.25 to 1. It is the object of this aspect ratio for the user to push the film into the intended area of use facilely and also be narrow enough to avoid sticking to the teeth by remaining within the buccal pouch between the inner cheek and gums or behind the upper or lower lips in front of the gums. It is also desirable that the product not be visible to third parties when sitting in the buccal pouch, which is more likely to occur as the top of the film reaches the tooth line. Conventional films for oral use are rectangular or square shaped with typical length by width ratios no greater than 1.5 to 1. In contrast, the films described herein can have a minimum length by width ratio of 2 to 1 or higher.

It is an object of the invention to provide for a film or sheet with rounded corners to minimize sharp corners and reduce the perception of stiffness when placed in the upper or lower lip or buccal cavity. It is a further object to provide caplet shaped films to further provide for rounded corners.

It is an object of the invention to provide for a film or sheet that wherein multiple said films or sheets may be packed loosely in a container without individual film/sheet packages. It is a further object of the present invention that films or sheets do not tackiness together when stored at ambient conditions. In certain embodiments, the films or sheets do not tackiness together when the packaging container with loose films is subjected to 30 days of 25 C/60% relative humidity (RH).

Stability of the film or sheet can be thought of in a number of ways. These include chemical stability of the active ingredient, as well as physical stability of the dosage form itself. Physical stability can be thought of in different ways: the film or sheet retaining its form, i.e. not curling, bending or melting or otherwise deforming; the propensity of the film or sheet to increase in moisture (typically considered under accelerated stability conditions), and when relevant to the packaging configuration, propensity of films or sheets to increase in tackiness and tend to stick together.

In certain embodiments, the film or sheet maintains its form without deforming after 5 days, preferably 10 days, more preferably 30 days at one or more of the following conditions: 25 C/60% relative humidity (RH); 30 C/65% RH; 30 C/75% RH; or 40 C/75% RH.

In certain embodiments, the films or sheets, when packaging in a multi-film/sheet chamber, do not stick together and/or to container surfaces after 5 days, preferably 10 days, more preferably 30 days at one or more of the following conditions: 25 C/60% relative humidity (RH); 30 C/65% RH; 30 C/75% RH; or 40 C/75% RH.

Five days is considered a reasonable time in which to consume the contents of a unit dose container after removing an overwrap. In other cases, a user will consume the fill contents within two days, in which case the moisture uptake discussed above may be considered in a forty-eight-hour period.

It is an object of the present invention to provide for a film or sheet capable of releasing perceptible levels of flavor and/or sweetener, when used in the buccal cavity, sublingual cavity, supralingually, or in the upper lip, for greater than ten minutes, preferably greater than fifteen minutes, more preferably greater than twenty minutes, and most preferably for greater than thirty minutes.

Nicotine compositions will generally range from 1 mg per film or sheet, to up to 30 mg per film or sheet. A preferred nicotine dose range is 2 mg to 12 mg per film or sheet.

The film or sheet may comprise polymers of 50% or greater, preferably 75% or greater, more preferably 85% or greater, most preferably 90% or greater particularly (though not exclusively) where the film or sheet contains a nicotine active ingredient.

Cannabinoid compositions will generally range from 5 mg per film or sheet, to up to 50 mg per film or sheet. A preferred cannabinoid dose range is 10 mg to 30 mg per film or sheet.

In embodiments, the films described herein allow constant (or substantially constant) release of an active agent (e.g., nicotine) through the mucosa of a user.

In aspects, the active agent is released as the film dissolves for a period of about, for example, 5 minutes, 6 minutes, 7 minutes, 8 minutes, 9 minutes, 10 minutes, 12 minutes, 15 minutes, 20 minutes or longer.

### BRIEF DESCRIPTION OF THE DRAWINGS

The accompanying drawings illustrate aspects of the present invention. In such drawings:

FIG. 1 displays a side view of a paddle wheel according to embodiments of the invention.

FIG. 2 is a perspective view of the paddle wheel embodiment of FIG. 1.

FIG. 3 is a cross-sectional view of a paddle wheel embodiment.

FIG. 4 is a cross-sectional view of a paddle wheel embodiment along line 4-4 of FIG. 3.

FIG. 5 is a perspective view of a turbofan embodiment of the present invention.

FIG. 6 is a top view of a turbofan embodiment of the present invention.

FIG. 7 is a perspective view of a turbofan embodiment without a cylindrical case present.

FIG. 8 is a cross-sectional view of a turbofan embodiment along line 8-8 of FIG. 7.

FIG. 9 is a cross-sectional view of a turbofan embodiment of the present invention.

FIG. 10 is a perspective view of a multi-chambered rotating cassette which includes arrowhead-shaped features to center the film position within the chamber.

FIG. 11 is a top view of a multi-chambered rotating cassette which includes arrowhead-shaped features to center the film position within the chamber.

FIG. 12 is a perspective view of a shaped film or sheet intended for use over teeth, with approximately 90-degree angles at two bend points.

FIG. 13 is a perspective view of a shaped film or sheet intended for use over teeth, with an approximately continuous curve.

FIG. 14 is a perspective view of a shaped film intended for use over teeth, with a non-continuous curve.

FIG. 15 is a flow-chart of steps in dynamic roll molding according to certain embodiments of the present invention.

FIG. 16 is a perspective view of the lower component of a puck with a conical insert. The insert in FIG. 16 is smooth (not scalloped like FIG. 17). The user can slide a film up the cone to a point where the top of the film is presented with two sides of the top of the film available to be grabbed by the user. The optional flat top of the cone makes more of the film presented when slid up. The conical insert (which may be a separate piece from the bottom of the puck, or a separately made piece that is inserted) optionally features holes that form air connections to space at the bottom of the conical insert, which space may optionally include a desiccant.

FIG. 17 is a perspective view of the lower component of a puck with a conical insert. The insert is scalloped, with a series of adjoining sections that are optionally concave as depicted in FIG. 17. The scalloped feature allows the user to slide the film up, to a point where the film is presented with the upper end of the film above the center of the cone where it can be grabbed easily. As with FIG. 16, optional holes form air connections to space at the bottom of the conical insert, such space to optionally include a desiccant.

FIG. 18 is a side perspective of the lower component of a puck with a conical insert shown in FIG. 16.

FIG. 19 is a perspective view of a multi-chambered rotating cassette which includes arrowhead-shaped features with an outer case and aperture.

FIG. 20 is a perspective view of an outer case of the multi-chambered rotating cassette of FIG. 19.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

Nicotine refers a naturally produced alkaloid in the nightshade family of plants (most predominantly in tobacco and Duboisia hopwoodii) and is widely used recreationally as a stimulant and anxiolytic. As a pharmaceutical drug, it is used for smoking cessation to relieve withdrawal symptoms. Nicotine acts as a receptor agonist at most nicotinic acetylcholine receptors (nAChRs), except at two nicotinic receptor subunits (nAChRα9 and nAChRα10) where it acts as a receptor antagonist.

The term “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

The term “an effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin or muscles.

The terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

The term “oral thin film” or “OTF” refers to a thin film composed of a mono or multi-layer polymeric matrix that is applied in the mouth. OTFs can be water-based soluble polymers designed to rapidly deliver drugs systematically. Depending on the drug candidate and the targeted indication, OTF can be formulated either as orodispersible films or a transmucosal film.

The term “tackiness” generally refers to the proclivity of one film or sheet to adhere to another surface.

The terms “film” or “sheet” or “oral film” or “oral sheet” or “oral film product” or “oral sheet product” or “oral drug product” or “film product” or “sheet product” or “drug product” or “product” can be used interchangeably.

Orodispersible films work like conventional orodispersible tablets. In this case, OTF dissolves within seconds in the mouth and the drug substance is swallowed together with the saliva and takes the same route as a tablet. With a transmucosal film, the active substance directly enters the systemic circulation via the buccal mucosa, avoiding the hepatic first-pass effect.

The active ingredient can be either dissolved or suspended in the polymeric matrix. The OTF dosage form presents several advantages, among which: (a) an easy drug intake therefore high acceptance by young and old patients (b) no swallowing difficulties and no beverages requirement for intake, (c) flexible manufacturing with area proportional dosage (different doses from one base bulk), (d) potential avoidance of the first-pass effect and improved bioavailability, (e) innovative and appealing design offering more options for differentiating a product range and (f) a discreet and pain-free application.

The term “Soluplus®” or “SOL” refers to an amphiphilic graft copolymer studied as a micellar solubilizer for drugs. SOL is a unique water-soluble graft copolymer constructed from hydrophilic and lipophilic portions. The hydrophilic moiety is composed of PEG residue corresponding to 13% of the polymer, whereas the polyvinyl caprolactam and polyvinyl acetate act as the lipophilic backbone in the polymer structure. This amphiphilic polymer can be used above the critical micelle concentration (CMC) to dissolve certain drugs via micelle formation. However, below CMC, SOL is used to inhibit drug precipitation via restraining the drug nucleation and crystal growth and to provide steric stabilization in a supersaturated state.

The term “hydroxypropyl cellulose” or “HPC” refers to a derivative of cellulose with both water solubility and organic solubility. It is used as an excipient, and topical ophthalmic protectant and lubricant. HPC is an ether of cellulose in which some of the hydroxyl groups in the repeating glucose units have been hydroxypropylated forming —OCH₂CH(OH)CH₃ groups using propylene oxide. The average number of substituted hydroxyl groups per glucose unit is referred to as the degree of substitution (DS). Complete substitution would provide a DS of 3.

The term “hot extrusion” refers to the act of extruding an article, while the article is currently being heated, or was previously heated, such that the article is at an elevated temperature during the extruding process. The extruding process typically includes forcing the article through a die, thereby obtaining a desired cross-section.

The term “casting” or “film easting” refers to the act of removing liquid (e.g., water and/or solvent) from a mixture (e.g., slurry), such that a film is produced.

The term “condensing” refers to the act of removing liquid (e.g., water and/or solvent).

The term “packaging material” refers to those materials and substances employed to package the product (e.g., thin film). Such materials are widely known to those of skill in the art.

The term “enclosing” refers to the packaging materials containing or holding the product (e.g., thin film) by surrounding the product with the packaging material. The packaging materials can partially surround the product or can completely surround the product. Typically, to ensure safety (e.g., no tampering with product) and freshness, the packaging materials will completely surround the product. For example, the packaging materials can form a relatively vapor impermeable enclosure of the product.

The term “low surface energy” or “LSE” refers to substrates or plastics made of materials such as vinyl, polyethylene and polystyrene and have unique surface characteristics making them harder for pressure sensitive adhesives to stick. These plastics are generally soft and have low density which helps in lightweighting applications.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the methods and systems particularly pointed out in the written description and claims hereof.

### Dynamic Roll Molding

In aspects, the film or sheets described herein are produced by dynamic roll molding. “Dynamic roll molding,” refers generally to (1) milling and mixing the feedstock with or without spraying of liquid components; (2) pelletizing the feedstock; (3) softening the feedstock at processing temperatures and passing the feedstock through rollers, die or orifice; (4) controlling the thickness resulting material by stretching and rolling (and adding optional features like texture, embossing, debossing), and (5) die-cutting individual pieces. For most embodiments of the present invention, the final product is a film or sheet.

In dynamic roll molding, the initial feedstock is preferably a substantially non-aqueous feedstock. By substantially non-aqueous, it is meant that the only water is that of moisture that is typically present in the compositional ingredients. Water may be optionally included in non-preferred embodiments; however, water is generally not desired in the final composition.

In dynamic roll molding, the initial feedstock is a free-flowing fine powder mixture or a granulated powder mixture. The initial feedstock may be produced by direct blending of excipients and drugs in a V-blender type mixer with an i-bar with or without a spray capability; or by spray granulation; or high shear granulation; or spray drying; or spray congealing; or low shear granulation; or microencapsulation. The feedstock may then be further wet or dry granulated by pelletizing or roller compaction and milling.

In dynamic roll molding, the initial feedstock is comprised of a mixture of excipients and actives wherein some excipients melt at processing temperatures and other excipients may be non-melting. The system is may therefore be a biphasic mixture of flowing molten excipients and non-molten phase distributed within.

In other embodiments, the initial feedstock is comprised entirely of non-molten excipients which only soften during processing above their glass transition temperature but below melting temperature.

In other embodiments, the initial feedstock is comprised entirely of molten excipients or excipients which soften during processing above their glass transition temperature but below melting temperature.

In dynamic roll molding, the initial feedstock is subject to heat-typically above the glass transition temperature and below the melting point such that it is adequate to soften the feedstock. In preferred embodiments, the process heat is below the melting point of all of the feedstock constituents (except for those ingredients that are liquid at room temperature. Preferably, the process heat is below the melting point of 95% of the non-liquid ingredients, measured by weight; less preferably the process heat is below the melting point of 90% of the non-liquid ingredients, measured by weight.

Preferably, processing temperatures for softening do not exceed 120° C., preferably do not exceed 110° C., more preferably do not exceed 100° C., and most preferably do not exceed 90° C. Low processing temperatures are particularly useful for nicotine oil, which tends to sublime. Low processing temperatures are also useful for temperature sensitive actives.

However, where active ingredients are not heat sensitive, higher processing temperatures may be employed, i.e., 150° C. to 400° C., preferably 175° C. to 250° C., wherein a majority fraction of the excipients are molten.

After optional softening, the feedstock is then initially shaped, typically using a die and/or rollers. The dough like semi-molten feedstock may be extruded or passed through rollers. Optionally the material is shaped by the die preferably into a filament or rope, but other shapes like films, sheets or tubes are expressly contemplated.

The resulting material is then compressed through heated and/or unheated rollers to produce a film or sheet of the desired thickness. Additionally, the rollers, or an additional set of heated or unheated rollers, may be used to texturize the film or sheet stock. Additionally, rollers may be employed to emboss product strengths, brands, or designs or shapes on the film or sheet stock.

Rollers may be used to maintain uniform thickness of the sheet or film. Preferably, the thickness ranges +/−10%; more preferably +/−7%, most preferably +/−5%. Rollers may be at ambient temperature, actively warmed, or actively chilled. The feedstock may be optionally pre-heated or chilled prior to being passed between rollers.

The film/sheet is cut into individual doses. This may involve slitting where the width of the web is wider than the desired dose. The dose may be cut or die cut. Die cutting is desirable where rounded edges or non-rectangular shapes are desired.

Die cutting surplus material can be recycled, i.e., run through the process. Preferably the surplus material may be shredded into fine pieces in an appropriate type of shredder and mixed with the powdered feedstock.

An extruder is an example of equipment that can be used for softening or molding the feedstock.

A heat jacketed high or low shear granulator is an example of equipment that can be used for softening the feedstock.

Dynamic roll molding can achieve content uniformity with a deviation of the intended target loading of active ingredients by +/−10%, preferably +/−7.5%, more preferably +/−5%, and most preferably +/−3%.

Applicants have found that ordered mixing is desirable prior to the softening process, particularly where the active ingredient is an oil. Ordered mixing is particularly desirable when working with viscous materials like cannabinoid distillates.

While high shear mixing may be employed, generally low shear mixing is adequate.

Where the active ingredient is viscous, it may be desirable to mix the active ingredient with another liquid to thin the composition. Without limitation, medium chain triglycerides are suitable for this purpose; glyceryl monooleate or triglyceryl monooleate is also suitable for certain embodiments.

Dispenser with Paddle Wheel Design

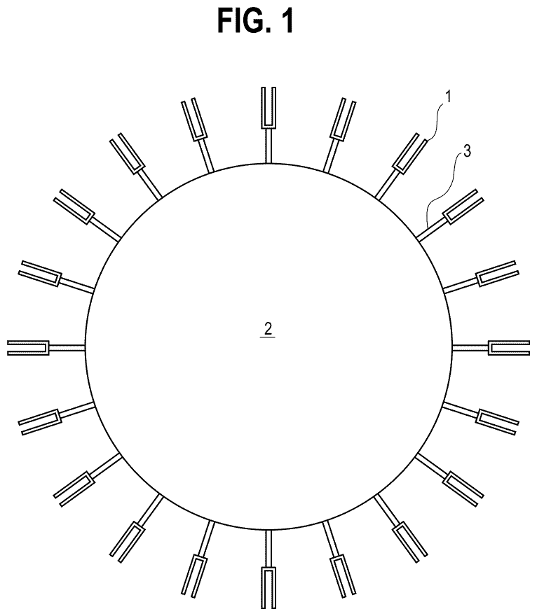

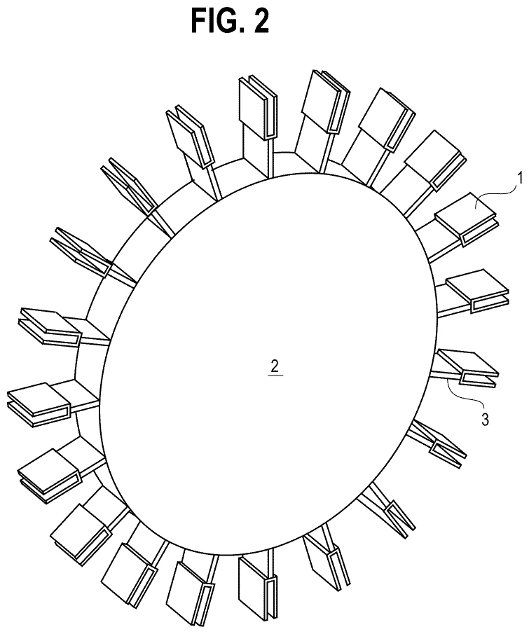

One embodiment of the invention relates to a paddle wheel design. FIG. 1 and FIG. 2 depict a side view and perspective view of a paddle wheel configuration. The container/dispenser of this embodiment can include a hub 2 with spokes 3. Extending from the spokes are flaps 1 which act to hold a film or sheet in place until rotated in alignment with an aperture for dispensing. The flaps of this embodiment incorporate a paddle design which functions as a container for one film or sheet. The paddle can include two parallel flaps, a pitchfork configuration, or flaps that are connected on one or more edges that hold a film or sheet.

The hub can be rotated to align a given paddle wheel chamber with an aperture (which itself may be required to be opened by the user). The film or sheet can then be removed from the paddle wheel chamber through the aperture. In certain embodiments, the film may simply drop out by the force of gravity at a predetermined time, interval, or orientation. In certain embodiments, the film may be shaken or agitated out. The aperture can be located on any surface of the container and multiple apertures are also contemplated.

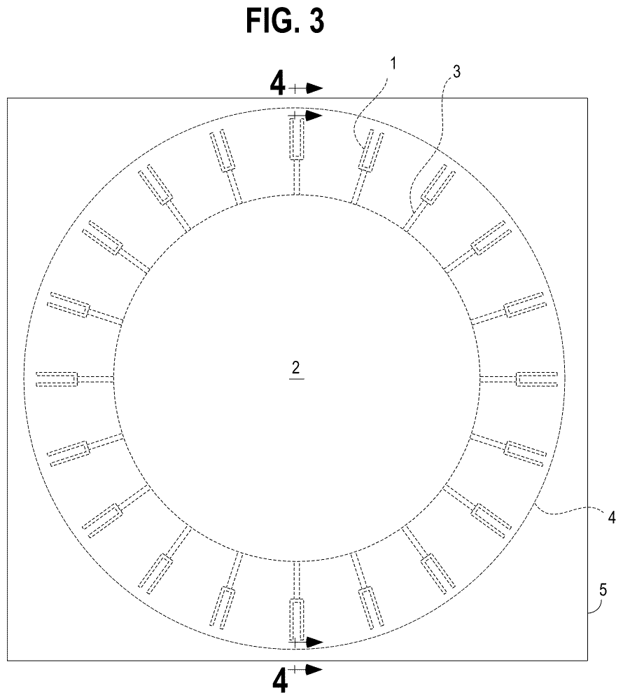

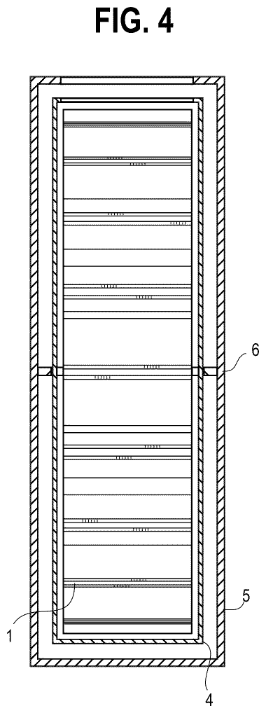

As depicted in FIG. 3 and FIG. 4, the individual film chambers created by the flaps can form part of the outer edge of the wheel. In further aspects, the paddle wheel chambers are placed within an outer cylinder 4, and are able to rotate within the outer cylinder. The outer cylinder may be used to prevent films or sheets from exiting their individual chambers until those individual chambers reach the correct aperture.

In certain embodiments, an outer case 5 may feature a sliding tab or tray built into it which slides (or otherwise moves) outward from an original position closer to the hub (not shown). When the cassette is rotated along an axle 6 such that a film-loaded chamber is aligned at an aperture, the sliding tab may be engaged to push the film out of the chamber. The sliding tab and the paddle wheel walls may optionally be designed such that they would prevent the cassette from rotating to the next chamber until the sliding (or otherwise moving) tab is returned to its original position.

In certain embodiments, the outer case includes a sliding tray with a bump or protrusion that engages with a notch on the film edge to drag out the film from the chamber when the tray is opened by sliding outward. Similar notches can also be built into the chamber walls so that the cassette cannot rotate when the tray is open.

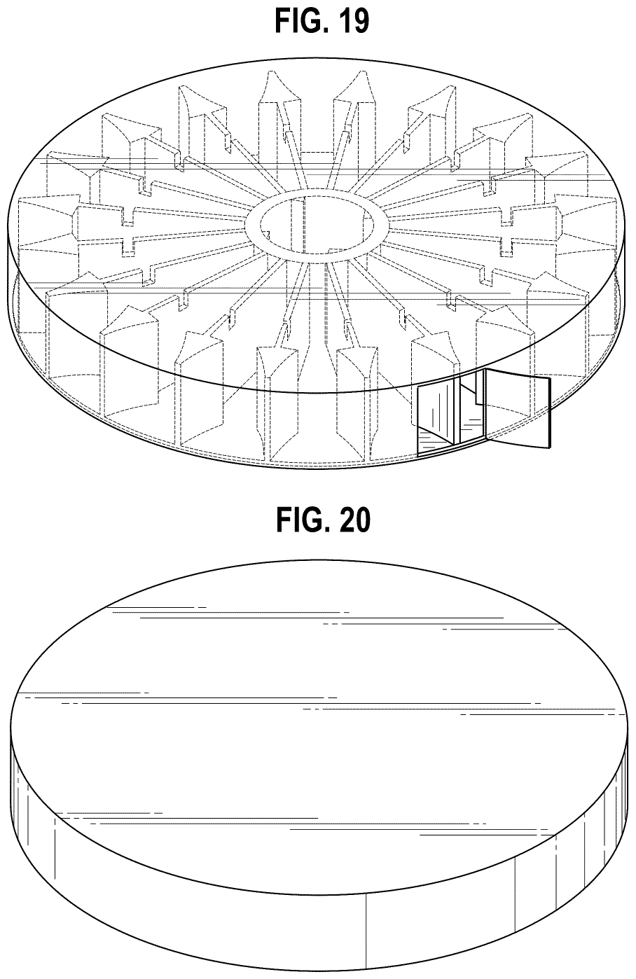

In yet other embodiments, the individual chambers in the round cassette of the paddle wheel design may have walls with features to keep the film standing on its edge and approximately centered within the chamber (or wherever desired). For example, as shown in FIG. 10 the chamber may feature arrowhead-shaped structures 11 affixed on the outer ends of the chamber walls. The arrowhead-shaped structures may also be sized and designed to prevent the sliding tray from being pulled out (or otherwise moved) from the dispenser, thereby acting as a stop. As shown in FIG. 19, the film or sheet is positioned such that the outer portion of the film extends beyond the narrow field created by the two arrowhead shapes. As a result, the gap created by the arrowheads introduces positional stability of the film or sheet. The arrowhead design is non-limitative; other geometric shapes may similarly be employed to create one or more narrow gaps that ensure positional stability. By “positional stability” we mean, the distance between the two arrowhead sides (or other shapes and/or barriers than retain the film or sheet) is slightly greater than the thickness of the film contained within. The arrowhead sides or other protrusion help center the film within the individual chamber such that the film is as close as possible to the center of the aperture.

Preferably, the arrowhead sides or other positional elements incorporated within the chamber are from 0.025 mm to 3 mm apart, more preferably 0.5 mm to 2 mm apart and most preferably 0.5-1 mm apart. This the range of motion in the horizontal direction for the film or sheet in the chamber—assuming the cassette is flat with respect to the ground—is limited by the positional elements or the arrowhead sides.

In certain embodiments of the round cassette or container of the invention, the segment at the bottom of the individual chamber has a rounded, flat or curved aspect to prevent the film from getting wedged within the narrowing bottom part of chamber.

In certain embodiments, the cassette or container includes a spiral or other eccentric cam on the central hub which acts to push a film out radially when the film reaches an aperture opening (not shown).

In another embodiment, a restraining pin or plate is built into the case near the aperture and protrudes into the rotatable cassette such that the rotatable cassette cannot advance to the next position until the film aligned at the aperture is removed (not shown).

In further aspects, the outer cylinder is situated in an outer case. The outer case may be rectangular in shape, but other shapes known in the art and suitable for the uses of the present invention are contemplated.

The paddle wheel may hold 1-40 films or sheets, but may hold as many as 100 to 200 sheets or more.

Dispenser with Turbofan Design

FIG. 5 depicts a container/cassette that includes a push-button actuator 9 with longitudinally situated chambers 8 that project radially from a centrally located hub. A cylindrical case 10 houses the container/cassette, although myriad shape configurations for both the container/cassette and case are contemplated, including spherical, pyramidal, prismatic, cuboid, cube, elliptical, arrowhead shaped, or any other shape or configuration known in the art.

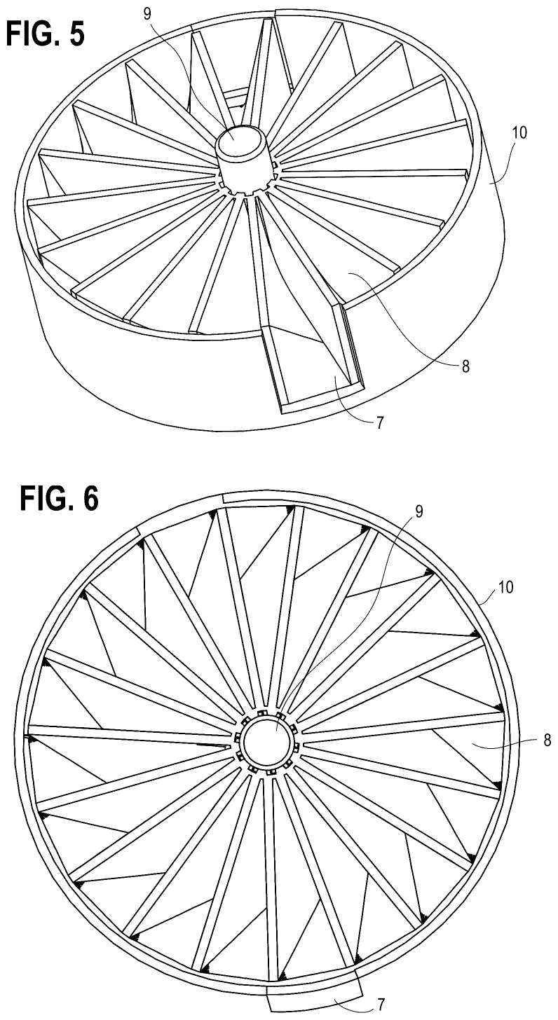

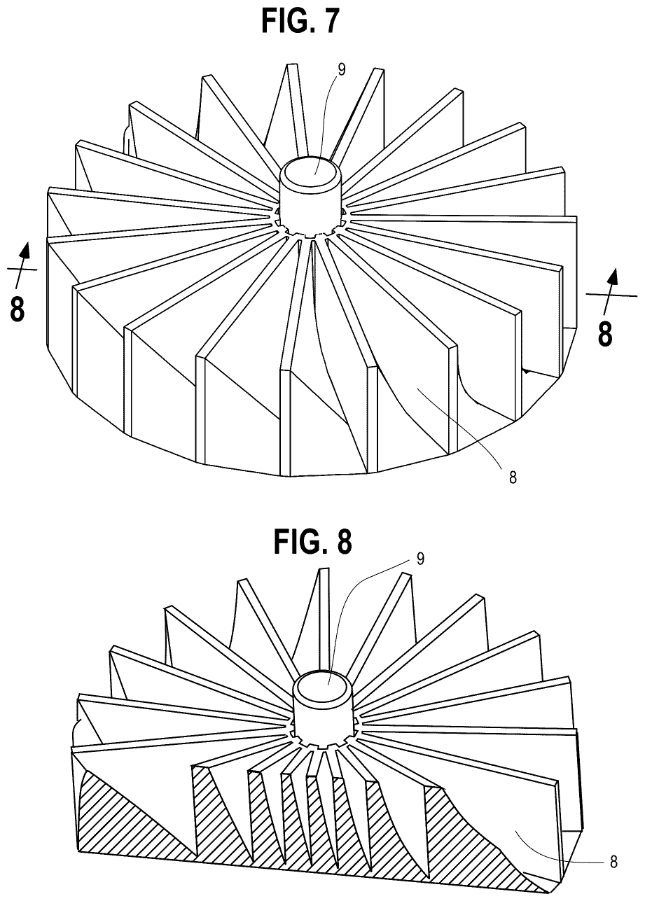

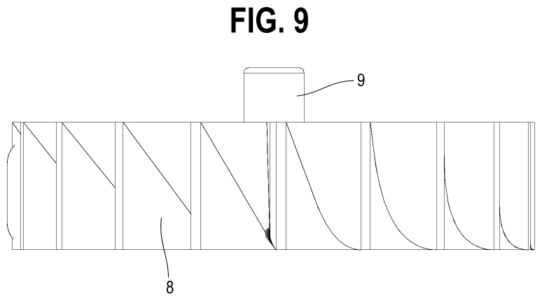

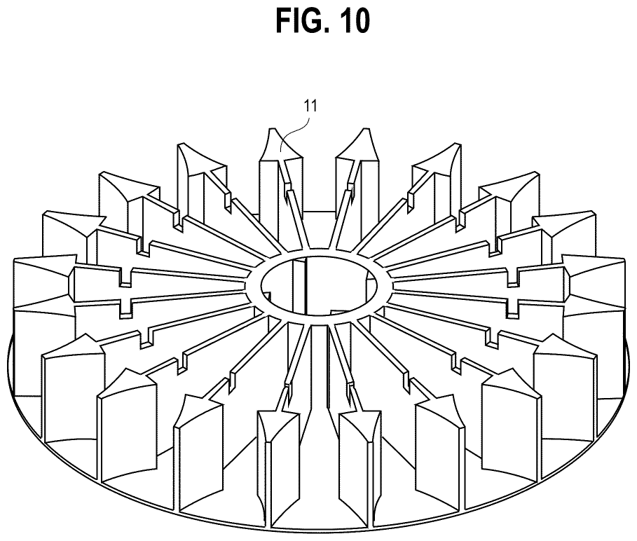

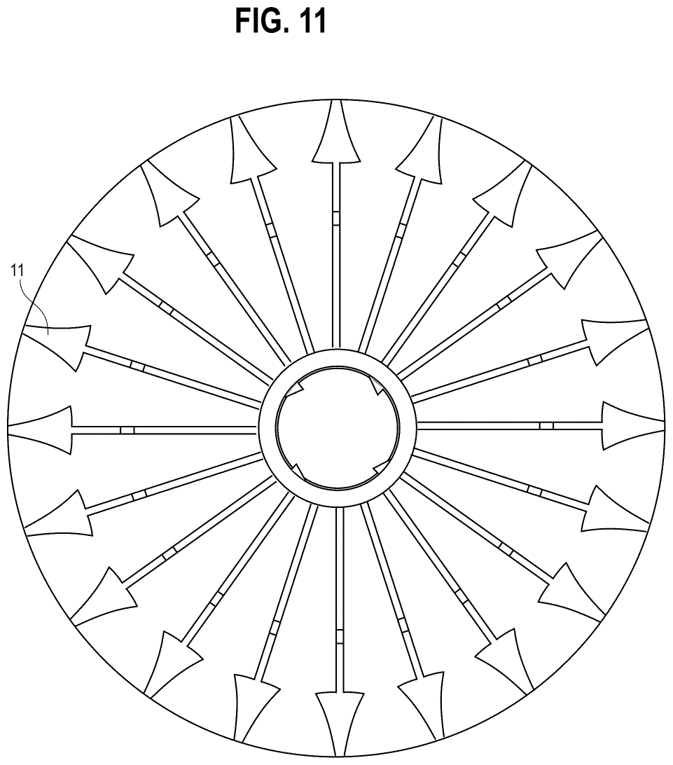

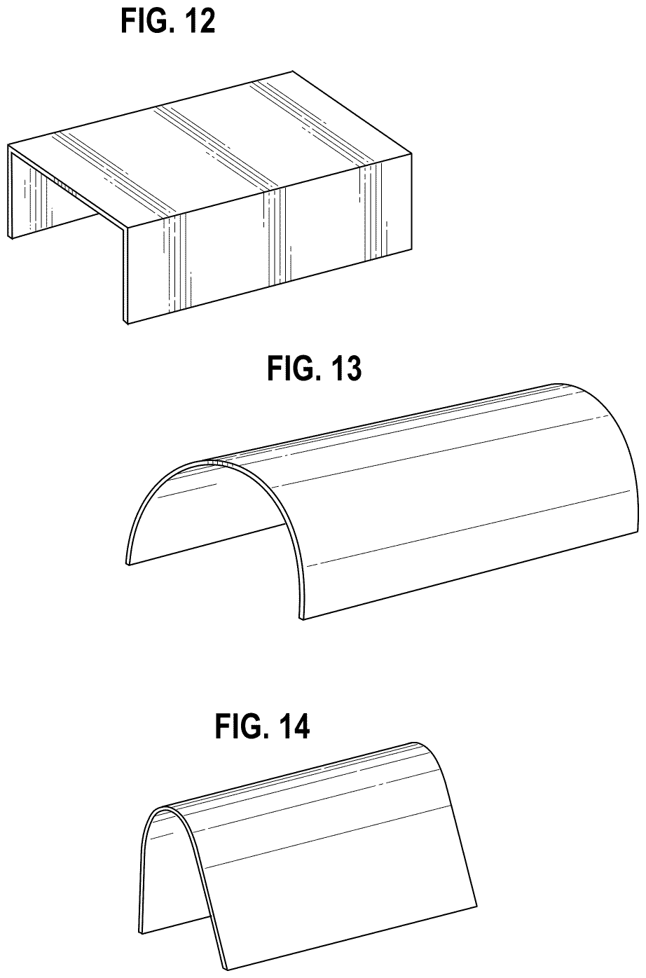

As shown in FIG. 5-FIG. 8, the chambers within the container may be angled, such that the configuration of the device resembles a turbofan of an engine. The contained films or sheets—when the container is resting flat—may sit at an angle in the chamber which is equal in magnitude and direction to the angle of the chamber itself. In aspects, each chamber holds one film or sheet. In other aspects, each chamber holds multiple films or sheets.

In aspects, the film or sheet rests longitudinally in the chamber. In certain embodiments, the film or sheet length is at least 50% of the length of the chamber. In other embodiments, the film or sheet length is at least 65% of the length of the chamber. In yet other embodiments, the film or sheet length is at least 80% or more of the length of the chamber.

In certain embodiments, the height of the chamber containing a film or sheet is no more than 110% of the height of the film or sheet. In other embodiments, the height of the container containing a film or sheet is no more than 120% of the height of the film or sheet.

The container may be placed within a cylindrical case so that films or sheets cannot fall out of their chambers until they reach the aperture.

In certain embodiments, the chambers are not capable of rotation within a case or container. Alternatively, the chambers remain fixed and a top or bottom cover is supplied that is capable of rotation on the case or container such that the rotation reveals the films or sheets within a given chamber.

### Chamber Surfaces

Formulating the films to be non-tacky not only prevents films from sticking to one another but also prevents film adhesion to packaging surfaces. When coupled with film properties of stiffness and dimensional stability, the film or sheet can be calibrated to present itself at the center of the wheel package aperture and the reduced tackiness allows for easy removal from the package. This is also assisted by wheel chamber design that minimizes contact points between the film and packaging surfaces and also centers the film within the chamber to prevent it from falling flat or near flat in the chamber.

Prevention of adhesion to packaging surfaces is important for effortless removal of the film by gravity wherein simply orienting package with aperture facing down would allow a film to slide out of the wheel chamber without tapping or with gentle wiggling or tapping.

The chamber surface may include various features to promote efficient release of a film or sheet or multiple films or sheets. The chamber surface may prevent sticking of films or sheets to one another in a multiple film or sheet chamber, by keeping them apart and/or promoting movement of the films or sheets. The chamber surface may prevent sticking of films or sheets to the surface of the container. The chamber surface

Accordingly, the surfaces of the container may be non-smooth and include a rough texture to retard the sticking and tackiness of the films or sheets. The surfaces of the container may be pocked with semi-spheres or other geometries. A grate or grilled surface may be employed to reduce contact surface area with a film or sheet or with multiple films or sheets.

In embodiments, 50% or more of the chamber surface area may be non-smooth, rough or pocked. In other embodiments, 80% or more of the chamber surface area may be non-smooth, rough or pocked. In other embodiments, 95% or more of the chamber surface area may be non-smooth, rough or pocked.

The contact surface area of the container may include a low-surface-energy substrate that possesses a surface energy of 40 dynes/cm or less. In other embodiments, the contact surface area of the container may include a low-surface-energy substrate that possesses a surface energy of 30 dynes/cm or less.

In other embodiments, the contact surface area of the container may include a low-surface-energy substrate that possesses a surface energy of 20 dynes/cm or less.

In embodiments, more than 50% of the chamber surface area may include low-surface-energy substrate. In other embodiments, more than 75% of the chamber surface area is composed of low-surface-energy substrate. In preferred embodiments, more than 95% of the chamber surface area is composed of a low-surface energy substrate.

In certain embodiments, the chambers may include a number of protruding pins which act to minimize the extent of contact between package surfaces or the extent of contact between films or sheets with package, chamber, or container surfaces. Similarly, brushes, fibers or filaments may be used in the chamber(s) to minimize the extent of contact between package surfaces or the extent of contact between films or sheets with package, chamber, or container surfaces.

In further embodiments the film product may have a notch cut along one or both edges to allow a corresponding protrusion in the package to sit within the notch and prevent the film from falling out of the chamber unless the protrusion is disengaged by a suitable mechanism built into the container. This prevents a film from falling out even if the aperture were always open unless the user actively interacts with the mechanism restraining the film within the chamber.

### Film Compositions

Various types of excipients and active ingredients are discussed herein. Such lists are expressly non-limitative.

### Film Formers

A preferred film former is hydroxypropyl cellulose (HPC), preferably a low molecular weight HPC (80,000 daltons or fewer), more preferably 60,000 daltons or fewer, most preferably 40,000 daltons or fewer. A preferred grade is HPC ELF.

Another preferred film former is Soluplus®, a polyvinyl caprolactam-polyvinyl acetate-polyethylene glycol graft co-polymer.

One way to measure dimensional stability is to look whether the film or sheet when placed on a flat surface bends in either of the length direction or the height direction after exposure to humidity and temperature. The bending occurs by upward or down curl of one or both ends of the film along the long axis or the short axis. This is considered after any substrate, overwrap or seam tape or film is removed. For example, after 24 hrs exposure to 30 C, 65% RH, does the film or sheet bend more than 5% in either of the length or height direction. If the film stays completely flush with the flat surface on which it is placed or visually bends less than 5%, we say the film or sheet has dimensional stability. Preferably, such dimensional stability also occurs after 48 hrs of such exposure; more preferably after 72 hours of such exposure and most preferably after. Applicants have similarly determined that such dimensional stability can be achieved with the use of corn syrup solids as disclosed herein.

Even when packaged with a moisture barrier overwrap, or covered seams, embodiments of the present invention will involve moisture exposure before use (e.g. remove overwrap, and duration of use of the container until the final film or sheet.

Applicants have determined that Soluplus is effective for promoting dimensional stability of the film or sheet. However, if the amount of Soluplus is too high, brittleness will be introduced.

A desired range for Soluplus, particularly for films or sheets made using dynamic roll molding, is 2-20% of the composition, preferably 6-16% most preferably 8-14%.

Applicants have found that HPC and Soluplus are effective film formers, particularly for films or sheets made by dynamic roll molding, and particularly for films containing nicotine and other oily actives. HPC/soluplus films or sheets have good mouthfeel properties and reduce the perception of gumminess of the films. Other actives are contemplated as well.

When used together, HPC typically used in greater amounts than Soluplus. A preferred ratio is from 1:10 (1 part Soluplus to 10 parts HPC) to 1:2 (1 part Soluplus to 2 parts HPC), preferably, Soluplus to HPC from 1:9 to 1:3 most preferably from 1:8 to 1:4.

Soluplus may be used with a cellulosic polymer. The cellulosic polymer is typically used in greater amounts than Soluplus. A preferred ratio is from 1:10 (1 part Soluplus to 10 parts cellulosic polymer) to 1:2 (1 part Soluplus to 2 parts cellulosic polymer), preferably, Soluplus to cellulosic polymer from 1-9 to 1-3 most preferably from 1-8 to 1-4.

Another preferred film former is Copovidone or Copolyvidone, a copolymer of 1-vinyl-2-pyrrolidone and vinyl acetate also known commercially as Kollidon® VA 64. A preferred ratio of HPC to Copovidone is 8:1 to 4.1, preferably from 7:1 to 5:1, most preferably 6:1.

Another preferred film former is 37erpinene37yrrolidone (PVP).

The polymer included in the films may be water-soluble, water-swellable, water-insoluble, or a combination of one or more either water-soluble, water-swellable or water-insoluble polymers.

The polymer may include cellulose or a cellulose derivative. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, 37erpin gum, polyacrylic acid, methylmethacrylate copolymer, carboxyvinyl copolymers, starch, gelatin, and combinations thereof.

Polyethene oxide (PEO) is a preferred polymer.

Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. Water insoluble polymers may be of particular use for specialized applications, for example, for extended disintegration time.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. Desirably, the water-soluble polymers are water-soluble or water-swellable having at least 20 percent by weight water uptake. Water-swellable polymers having a 25 or greater percent by weight water uptake are also useful. In some embodiments, films formed from such water-soluble polymers may be sufficiently water-soluble to be dissolvable upon contact with bodily fluids.

Other polymers useful for incorporation into the films include biodegradable polymers, copolymers, block polymers and combinations thereof. It is understood that the term “biodegradable” is intended to include materials that chemically degrade in the presence of a solvent, as opposed to materials that physically break apart (i.e., bioerodible materials). Among the known useful polymers or polymer classes which meet the above criteria are: poly(glycolic acid) (PGA), poly(lactic acid) (PLA), polydioxanes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. And are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers that provide mucoadhesive properties to the film or sheet, as well as a desired dissolution and/or disintegration rate. In particular, the time period for which it is desired to maintain the film or sheet in contact with the mucosal tissue depends on the type of active contained in the composition. Some actives may only require a few minutes for delivery through the mucosal tissue, whereas other actives may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods or time, such as up to several hours, which may be desirable for delivery of certain active components.

For instance, in some embodiments, the films may include polyethylene oxide alone or in combination with a second polymer component. In some embodiments, the films may include polymers other than polyethylene oxide. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. Other specific examples of useful water soluble polymers include, but are not limited to, polyethylene oxide (PEO), pullulan, hydroxypropyl cellulose, polydextrose, polyvinyl pyrrolidone, carboxymethyl cellulose, polyvinyl alcohol, sodium alginate, propylene glycol alginate, carrageenan, polyethylene glycol, xanthan gum, tragacanth gum, guar gum, acacia gum, 39erpin gum, polyacrylic acid, methylmethacrylate copolymer, poloxamer polymers, copolymers of acrylic acid and alkyl acrylate (available as Pemulen® polymers), carboxyvinyl copolymers, starch, gelatin, pectin, and combinations thereof.

Specific examples of useful water insoluble polymers include, but are not limited to, ethyl cellulose, hydroxypropyl ethyl cellulose, cellulose acetate phthalate, hydroxypropyl methyl cellulose phthalate, acrylic polymers, vinyl acetate, sodium sulphonated polyesters, carboxylated acrylics, trimethylpentanediol/adipic acid/glycerin cross polymer, polyglycerol-2-diisostearate/IPDI copolymer, carboxylated vinyl acetate copolymer, vinylpyrrolicone/vinyl acetate/alkylaminoacrylate terpolymers, vinylpyrrolidone/vinyl acetate copolymer, and combinations thereof.

In accordance with some embodiments, polyethylene oxide may range from about 20% to 100% by weight in the polymer component, more specifically about 30% to about 70% by weight, and even more specifically about 40% to about 60% by weight. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight.

The molecular weight of the polyethylene oxide also may be varied. In some embodiments, high molecular weight polyethylene oxide, such as about 4 million, may be desired to increase mucoadhesivity of the film. In some other embodiments, the molecular weight may range from about 100,000 to 900,000, more specifically from about 100,000 to 600,000, and even more specifically from about 100,000 to 300,000. In some embodiments, it may be desirable to combine high molecular weight (600,000 to 900,000) with low molecular weight (100,000 to 300,000) polyethylene oxide in the polymer component.

### Pore Formers

Pore formers are soluble organic materials to adjust the disintegration time of the film composition in the buccal cavity. The pore formers are typically incorporated in the film as non-molten components and remain dispersed as particles throughout the film matrix.

Preferred ratio of pore formers to film formers is 1:10 to 1:2, more preferably 1:8 to 1:3, most preferably 1:6 to 1:4.

Applicants have determined that corn syrup solids are highly useful for embodiments of the present invention. Corn syrup solids can be used to accelerate disintegration of the film or sheet. In addition, corn syrup solids have a low propensity to absorb moisture and can be useful to promote dimensional stability of the film or sheet. A preferred range of corn syrup solids is 5 to 20%, preferably 8% to 15%.

A preferred pore former is corn syrup solids. Applicants have found that corn syrup solids reduce the gummy mouth feel of the product in the buccal cavity.

Another non-limitative example of an ingredient that may function as a pore former is isomalt. A preferred range of isomalt is 5 to 20%, preferably 8% to 15%.

### pH Modifiers

pH modifiers may be used to control pH (acidic or base), including without limitation, sodium bicarbonate, potassium bicarbonate, sodium carbonate, potassium carbonate, calcium carbonate, dipotassium phosphate, potassium citrate, sodium phosphate, Formic Acid/Sodium Formate, Hydrogen Chloride/Potassium Chloride, Hydrogen Chloride/Glycine, Hydrogen Chloride/Potassium Hydrogen Phthalate, Citric Acid/Sodium Citrate, Acetic Acid/Sodium Acetate, Citric Acid/Disodium Hydrogen Phosphate, Citric Acid/Trisodium Citrate Dihydrate, etc., and any other such buffer system. The buffer system may be designed to dynamically control the pH of the product taking into consideration the effect of saliva during use, i.e., a dynamic buffer system.

While sodium bicarbonate and similar basic salts may be used, salts are not preferred for certain embodiments, particularly nicotine containing films. In preferred embodiments, the nicotine film does not comprise a sodium or a potassium as a pH modifier.

Meglumine has surprisingly been found to be an adequate pH modifier, with particular suitability for use with nicotine films, including nicotine films made by dynamic roll molding with a pH greater than 6, preferably greater than 7, more preferably greater than 8, most preferably greater than 9. Meglumine can be incorporated without deleterious effects to film properties, including physical stability, dimensional stability and targeted disintegration time.

A preferred range of meglumine concentration is 0.25-9%, preferably 0.4% to 5%, most preferably 1-3%. A preferred amount of meglumine per film or sheet is 0.25 mg to 14 mg, preferable 0.4 mg to 7.5 mg, most preferably 1 mg to 4 mg.

Other non-limitative examples of pH adjusters are triethanolamine, arginine and lysine. A preferred range for basic amino acids as pH modifiers, is 0.25-9%, preferably 0.4% to 5%, most preferably 1-3%.

Arginine is a preferred basic acid used in a range from 1.25% to 5%. In addition to pH, arginine counters the vasoconstriction caused by nicotine, thereby enhancing local blood flow and absorption, and thus may act as a permeation enhancer. Preferred amounts of arginine in an individual film or sheet are 1.25 mg to 6, preferably 1 to 3 mg.

Non limitative, examples of buffer systems to obtain a pH include dibasic sodium phosphate and monobasic sodium phosphate. Both are FDA accepted buffer materials used and listed in the inactive ingredients list. For example, for a pH of 7, the ratio of monobasic/dibasic can be 4.6/8.6; for a pH of 7.5 the ratio of monobasic/dibasic can be 1.9/11.9; and for a pH of 8.0 the ratio of monobasic/dibasic can be 0.6/13.4. These are mathematically calculated buffer numbers and will need to be adjusted according to the other ingredients added to the formula. They also need to be adjusted for the length of time designed for the dissolution of the dosage unit on the buccal mucosa since saliva can be of a pH of about 6.8 but as it is made in larger amounts in the mouth the pH of saliva can sometimes become more basic. Thus, this dynamic buffer range is adjusted in the dosage unit by the amount s of the buffer system since saliva is freshly renewable in the mouth. See U.S. Patent Application Publication Nos. 2009/0098192 A1 and US 2011/0318390 A1 discussing dynamic buffering and incorporated herein by reference. The dynamic buffer systems of the present invention may be acidic or basic.

### Surfactants and Penetration Enhancers

Several surfactants (surface active agents) and non-surfactant penetration enhancers have been found to improve the absorption of actives.

Applicants have found that meglumine acts as a penetration enhancer, particularly for nicotine. A preferred range of meglumine concentration is 0.25-9%, preferably 0.4% to 5%, most preferably 1-3%.

Gelucire may be used as a penetration enhancer as well as a surfactant. A preferred range of gelucire is 0.01% to 5%, preferably 01% to 2%, more preferably 0.075% to 0.35%. A preferred amount of gelucire per film or strip is 0.1 mg to 7 mg, preferably, 0.75 mg to 4.5 mg.

Applicants have found that sodium caprate is a useful absorption enhancer, including without limitation for cannabinoids. A preferred range for sodium caprate is 3% to 20%, preferable 5% to 15%.

Applicants have found triglyceryl monooleate (Caprol 3GO) to be a useful surfactant and absorption enhancer. A preferred range for Caprol 3GO is 0.5% to 5%, more preferably 1-4% and most preferably 2-3%.

Other penetration enhancers are expressly contemplated. A penetration enhancer is a substance that enhances absorption through the mucosa, mucosal coating and epithelium (otherwise known (see U.S. Patent Application Publication No. 2006/0257463) as a “penetration enhancer” or “permeability enhancer”). The mucosal absorbing enhancer may include but is not limited to polyethylene glycol (PEG), diethylene glycol monoethyl ether (Transcutol), 23-lauryl ether, aprotinin, azone, dextran sulfate, sodium caprate, salcaprazoate sodium, lauric acid, lauric acid/propylene glycol, lysophosphatilcholine, menthol, methoxysalicylate, oleic acid, phosphaidylcholine, polyoxyethylene, polysorbate 80, sodium EDTA, sodium glycholated, sodium glycodeoxycholate, sodium lauryl sulfate, sodium salicylate, sodium taurocholate, sodium taurodeoxycholate, sulfoxides, and various alkyl glycosides or, as described in U.S. Patent Application Publication No. 2006/0257463, bile salts, such as sodium deoxycholate, sodium glycodeoxycholate, sodium taurocholate and sodium glycocholate, surfactants such as sodium lauryl sulfate, polysorbate 80, laureth-9, benzalkonium chloride, cetylpyridinium chloride and polyoxyethylene monoalkyl ethers such as the BRIJ® and MYRJ® series, Kolliphors®, Gelucires®, benzoic acids, such as sodium salicylate and methoxy salicylate, fatty acids, such as lauric acid, oleic acid, undecanoic acid and methyl oleate, fatty alcohols, such as octanol and nonanol, laurocapram, the polyols, propylene glycol and glycerin, cyclodextrins, the sulfoxides, such as dimethyl sulfoxide and dodecyl methyl sulfoxide, the terpenes, such as menthol, thymol and limonene, urea, chitosan and other natural and synthetic polymers. Preferably, the mucosal absorbing enhancer is a polyol, e.g., polyethylene glycol (PEG), glycerin, maltitolsorbitol and other similar compounds.

Preferred surfactants include, without limitation, Pluronics, Kolliphors and propylene glycol.

Table 1 below sets forth additional surfactants which may be used in suitable amounts.

TABLE 1
Surfactants

| | | E-number/ |
|---|---|---|
| Surfactants | Acronym | Group |
| Lecithins | Lecithins | E 322 |
| Polyoxyethylene sorbitan esters | Polysorbates/Tweens | E 432-436 |
| Mono- and diglycerides of fatty | MDG/ | E 471 |
| acids | Monoglycerides | |
| Acetic acid esters of MDG | ACETEM | E 472 a |
| lactic acid esters of MDG | LACTEM | E 472 b |
| Citric acid esters of MDG | CITREM | E 472 c |
| Mono• and diacetyl tartaric acid | DATEM | E 472 e |
| esters of MDG | | |
| Sucrose esters of fatty acids | Sucrose esters | E 473 |
| Polyglycerol esters of fatty acids | Polyglycerol esters | E 475 |
| Polyglycerol polyricinoleate | PGPR | E 476 |
| Propane-l,2-diol esters of fatty | Propylene glycol | E477 |
| acids | esters | |
| Sodium stearoyl-2-lactylate | SSL | E 481 |
| Calcium stearoyl-2-lactylate | CSL | E 482 |
| Sorbitan fatty acid esters | Spans | E 491-495 |

### Plasticizers

The film and sheet composition can also include a plasticizer. The plasticizer may be present in an amount up to 30% based on the weight of the thermoplastic polymer. The plasticizer can be, without limitation, at least one of polyethylene oxide, polypropylene glycol, polyethylene glycol, glycerin, edible polyols, glycerol, polyols, maltitol, and reduced sugars.

Polyethelene oxide (PEO) is a preferred plasticizer, particularly where the product is intended to be packed loosely in a container. A preferred range of PEO is 0.01 to 10%, more preferably 0.05-2 most preferably 0.7-1.25%.

Where a conventional plasticizer is used, care must be taken to avoid product tack, particularly where the film product is packaged loosely. A preferred plasticizer range for plasticizers other than PEO is 0.5-5%.

A preferred plasticizer range for polyethylene glycol is 0.25 to 7%, preferably 1 to 3%.

### Mucoadhesive Agents

Mucoadhesive agents may be included to enhance mucoadhesion of the film or sheet. Mucoadhesive agents, may include, without limitation, propylene glycol, high molecular weight polyethylene oxides, sodium alginate, carbomer and polycarbophil.

A preferred range of mucoadhesive agents is 0.2-5%, preferably 0.5-3%, most preferably 1-2%.

### Oil Absorbent Agents

Oil absorbent agents are particularly relevant where oils are used in the composition, typically as one or more active ingredients.

Oil absorbent agents may include, without limitation, silicone dioxide, calcium silicate, magnesium stearate, magnesium silicate, soluble fibers (e.g., Polydex/Maltodextrin), and gums (e.g., Arabic, agar, etc).

A preferred range of oil absorbent agents is 0.2-10%, preferably 1-8%, most preferably 2-3%.

### Coloring Agents

A Coloring agent may be added to color the film or sheet. Food safe coloring agents are preferred. Colorants may include FD&C pigments or lakes as well as iron oxide pigments.

In preferred embodiments, the film/sheet is color-coded to flavor, e.g. an orange flavored film is orange in color, a cherry flavored film is red in color, a mint flavored film may be blue or green in color, etc.

### Flavor Agents

Flavor agents are typically employed. In addition, masking agents are useful where the active ingredient has a bitter taste profile. Bitter maskers or bitter blockers may be employed in effective amounts.

A preferred range of bitter blocker agents is 0.01-2%, preferably 0.1-1.5%, most preferably 0.2-1%.

### Sweeteners

Sweeteners are typically employed. Non-limitative examples include sucralose, glycyrrhizinates, acesulfame potassium, aspartame, neotame, neohesperidine, steviosides and monk fruit extract as well as natural sugars.

### Opacifiers

Any known opacifier may be employed to opacify the film or sheet (i.e., make the substance opaque and/or prevent/slow the rate of yellowing). Non-limitative examples include titanium dioxide and calcium carbonate. For certain embodiments, a clear film or sheet is preferred. For other embodiments, an opaque film or sheet is preferred.

Non limiting surfactants may include non-ionic surfactants, like polyol esters (e.g., glycol or glycerol esters, sorbitan derivatives); polyoxyethylene esters (e.g., polyethylene glycol (the “PEGs”; and poloxamers.

Applicants have found gelucire to be suitable for use as an anti-tacking agent. Low levels of Gelucires® 48/16 are employed, preferably less than 0.3% dry weight of the feedstock to prevent films packaged together from sticking to each other. Higher levels above 0.5% may tend to weaken the matrix undesirably.

Applicants have determined that medium-chain triglycerides (MCT Oil) is a useful excipient to retard tack and stickiness of certain embodiments, particularly formulations that comprise an oily active like nicotine oil which is soluble in water. This is particularly useful in multi film or sheet containers, and is also useful in single film/sheet chamber packaging.

MCT oil is used from 0.1% to 5%, preferably 0.5% to 2.5%, more preferably 0.75% to 1.5%, and most preferable 0.1% to 1.2%.

A combination of gelucire and MCT oil may be employed for anti-tacking.

### Glidants and Anti-Tacking Agents

Glidants may be employed to facilitated flow and feed of the feedstock.

Suitable, non-limitative examples of glidants include silicone dioxide and silicates like calcium silicate. Fumed silicone dioxide may be employed; a preferred grade is Aeroperl 300.

A preferred level of antitacking agents is 1-5%.

### Printed Indicia

The film or sheet stock is optionally printed with ink. Printed indicia may include active ingredient, strength, brand.

Alternatively, indicia may be applied through debossing/embossing. A preferred method is through rollers, which may be heated, chilled or ambient temperature.

Stabilizers, diluents, release modifiers, bulking agents, fillers, adhesives, anti-adherents, softeners, disintegrants, emulsifiers, elastomers may all be employed, preferably in effective amounts.

### Active Ingredients

Films or sheets of the present invention may include one or more active ingredients.

Nicotine compositions may comprise nicotine that is tobacco derived, or synthetic nicotine. It is expressly contemplated that any blend of stereoisomers, i.e., S-nicotine and R-nicotine. Nicotine polacrilex or similar ion exchange complexes may be employed; however, nicotine oil is a preferred embodiment, in part due to the low assay of nicotine polacrilex making higher nicotine loads harder to achieve.

Nicotine compositions may comprise free base nicotine, nicotine bitartrate, nicotine hydrochloride, nicotine benzoate, nicotine lactate or any other nicotine salt.

Certain actives, such as nicotine oil, may have a tendency to sublime off at elevated temperatures. For this reason, the use of a closed system to soften the composition may be desirable, i.e. the material is heated in an otherwise unvented system apart from the exit opening.

In addition, it may be desirable to overload the initial formula to provide for formulation loss. The initial formula be overloaded relative to the intended film dose by 10-50%, preferable 20-45%, most preferably 25% to 40%. Desirably, the peak processing temperature for nicotine oil is under 125 C, preferably under 110 C, more preferably under 100 C, and most preferably under 95 C. Such temperatures may similarly be employed with other actives prone to sublimation or degradation.

Other nicotine substitutes are contemplated, including without limitation, Imotine®. By nicotine substitutes, we mean molecules that are structurally similar to, but chemically different from nicotine. In many cases, nicotine substitutes like Imotine® act as an agonist at nicotinic acetylcholine receptors (nAChRs) (yet not nicotine). 6-methylnicotine may be employed as an active agent.

Nicotine and nicotine-substitute embodiments are contemplated for use as consumer products, as well as for use as pharmaceutical products (including smoking cessation and other non-cessation therapeutic uses).

Cannabinoid formulations may comprise phyto-cannabinoids as well as synthetic cannabinoids. Phyto-cannabinoids are typically derived from hemp, but may also be derived from Cannabis (i.e., marijuana) or from other plant sources. The cannabinoid may be present as plant material, a plant extract or essential oil, as a resin, a distillate or as an isolate. In certain cases the cannabinoid is solubilized in a carrier during the mixing phase.

Typically, the cannabinoid is present in a therapeutically effective amount.

In some embodiments, the Cannabis plant material provided to the composition is obtained from a Cannabis plant selected from the non-limitative group consisting of Cannabis sativa, Cannabis indica, and Cannabis ruderalis. Hemp may be used as a source.

In some embodiments, the composition provided herein includes a cannabinoid such as, for example, delta 9 tetrahydrocannabinol (Delta 9 THC), iso-tetrahydrocannabinol (iso-THC), delta 9 tetrahydrocannabinolic acid (Delta (THCA), delta 8 tetrahydrocannabinol (Delta 8 THC), delta 8 tetrahydrocannabinolic acid (Delta 8 THCA), cannabidiol (CBD), cannabidiolic acid (CBDA), cannabinol (CBN), cannabinolic acid (CBNA), cannabinol methyl ether (CBNM), cannabinol-C4 (CBN-C4), cannabinol-CZ (CBN-C2), cannabiorcol (CBN-C1), cannabinodiol (CBND), cannabigerol (CBG), cannabigerolic acid (CBGA), cannabigerolic acid monomethyl ether (CBGAM), cannabigerol monomethyl ether (CBGM), cannabigerovarinic acid (CBGVA), cannabichromene (CBC), cannabichromanon (CBCN), cannabichromenic acid (CBCA), cannabichromevarin (CBCV), cannabichromevarinic acid (CB CVA), tetrahydrocannabivarin (THCV), cannabidivarin (CBDV), cannabielsoin (CBE), cannabielsoic acid A (CBEA-A), cannabielsoic acid B (CBEA-B), cannabigerovarin (CBGV), cannabidiolic acid (CBDA), cannabidiol monomethyl ether (CBDM), cannabidiol-C4 (CBD-C4), cannabidivarinic acid (CBDVA), and cannabidiorcol (CBD-C1), cannabicyclol (CBL), cannabicyclolic acid (CBLA), cannabicyclovarin (CBLV), cannabitriol, cannabitriolvarin (CBTV), ethoxy-cannabitriolvarin (CBTVE), cannabivarin (CBV), cannabidivarin (CBVD), cannabitriol, cannabitriolvarin (CBTV), ethoxy-cannabitiolvarin (CBTVE), cannabifuran (CBF), dehydrocannabifuran (DCBF), and cannabiripsol (CBR), b-caryophyllene epoxide; mentha-l,8 (9)-dien-5-ol; pulegone; limonene; limonene oxide; a-terpinene; 50erpinene-4-ol; carvacrol; carvone; 1,8-cineole; p-cymene; fenchone; pulegone-l,2epoxide; b-myrcene; cannaflavin A; and cannaflavin B, or pharmaceutically acceptable salts thereof, solvates, metabolites, metabolic precursors, isomers or derivatives thereof.

Composites of cannabinoids are also contemplated; unrefined or semi-refined oils or extracts, including without limitation full spectrum oils and partial spectrum oils. Compositions can include purely synthetic cannabinoids or mixtures thereof. Synthetic cannabinoids are drugs that bind to endocannabinoid receptors and/or other receptors to which phytocannabinoids also bind. Typically, the cannabinoid is present in a therapeutically effective amount.

Synthetic cannabinoids may be of from any category such as classical cannabinoids, non-classical cannabinoids, hybrid cannabinoids, aminoalkylindoles, and eicosanoids. From a chemical structure classification viewpoint they may belong to the groups of Naphthoylindoles, Naphthylmethylindoles, Naphthoylpyrroles, Naphthylmethylindenes, phenylacetylindoles, Cyclohexylphenols, Tetramethylcyclopropylindoles, Adamantoylindoles, Indazole carboxamides and Quinolinyl esters. The cannabinoids may include any specific stereoisomer or a combination or racemic mixture.

The composition provided herein can include a cannabinoid such as, for example, various synthetic cannabinoid families such as the JWH series, HU series, CP series, AM series, AB series, AKB series, THJ series, UR series, XLR series, STS series, PB series.

The composition may non-limitatively include eicosanoids or endocannabinoids such as anandamide, methanandamide and 2-arachidonoylglycerol. The composition may include synthetic compounds that indirectly modulate the effect of phytocannabinoids or endocannabinoids by inhibiting their metabolism via fatty acid amide hydrolase (FAAH), Monoacyl glycerol lipase (MAGL) or other pathways. Non limitative examples include 4-nonylphenyl boronic acid, palmitoylsulfonyl fluoride, Cyclohexyl [I,G-biphenyl]-3-ylcarbamate.

In some embodiments, the film or sheet composition includes synthetic cannabinoids in combination with phytocannabinoids.

In certain embodiments, the film or sheet is sold as a dietary supplement, food, medical food, medical supplement or as a pharmaceutical. In some embodiments, the composition provided herein is formulated in the form a pharmaceutical composition, a nutraceutical, a cosmeceutical, a nutricosmetic, a cosmetic composition, a body care product, a personal hygiene product or a food product.

In preferred embodiments, an isolate is used which may be extracted and purified from plant material (i.e., any Cannabis or hemp varietal) or chemically synthesized. Preferred methods of extraction may be solvent or super-critical fluid extraction. Preferred methods of purification including conventional separation methodologies such as post-extraction liquid/liquid chromatography, or HPLC columns. Extraction techniques can also be calibrated to separate cannabinoids though typically post extraction separation is required for greater purity. The isolate is preferably 97% pure or greater, more preferably 98% pure or greater, and most preferably 99% pure or greater. Due to the expense of synthesized cannabinoid ingredients, film by deposit is a preferred manufacturing method due to its high yields (low waste).

In some embodiments, the cannabinoid is Delta 9 THC and/or CBD. In some embodiments, the cannabinoid is an anxiolytic cannabinoid such as CBD, THCA, CBDA and their esters for the treatment of generalized anxiety disorder, panic attack, PTSD, social anxiety disorder, phobias, separation anxiety disorder and premature ejaculation.

In some embodiments, the film includes, either in a single layer, or separately in different film layers, at least two cannabinoids. So, a film layer can include at least two cannabinoids, optionally with a non-cannabinoid layer. A bi-layer film can include a single layer with at least one cannabinoid, and a second layer with at least one cannabinoid.

In some embodiments, certain of which are described below, a film composition can include at least one cannabinoid, and at least one non-cannabinoid drug.

In some embodiments, a film composition includes at least one cannabinoid, and at least one non-cannabinoid bioactive agent. The non-cannabinoid bioactive agent can include any bioactive agent, where the bioactive agent is not a drug. A preferred embodiment includes a supplement combined within a single film composition with more than one cannabinoid to use or take advantage of the entourage effect between cannabinoids as well as a synergistic effect with non-cannabinoid supplements.

The entourage effect is a mechanism by which other cannabinoid compounds modulate or attenuate the overall psychoactive effects of delta 9 THC (or other forms of THC like delta 8 THC).

A preferred embodiment includes a drug molecule combined within a film or sheet composition with more than one cannabinoid to take advantage of the entourage effect between cannabinoids as well as a synergistic effect with non-cannabinoid drugs via different pharmacological mechanisms of action.

A preferred embodiment includes combining a drug molecule with delta 9 THC and cannabidiol to modulate or attenuate the psychoactivity of delta 9 THC.

A preferred embodiment includes combining a drug molecule with delta 9 THC and terpenes like pinene and limonene to modulate or attenuate the psychoactivity of delta 9 THC.

A preferred embodiment includes combining a drug molecule with delta 9 THC and cannabidiol and terpenes like pinene and limonene to modulate or attenuate the psychoactivity of delta 9 THC.

Other embodiments include breath freshener and oral health actives

Breath freshener active ingredients are well known, and in some cases simple flavors and sweeteners are used for breath freshening compositions.

A non-limitative list of active ingredients useful for breath freshening applications include: botanical extracts, and essential oils (anise, fennel, basil, mint, thymol, eucalyptol, menthol, spearamint, peppermint, tea tree, bergamont, cinnamon, clove, coriander, laurel, lavender, lemon, marjoram, mustard, orange, orris, parsley, pimento, pine, rosemary, sage, sassafras, thyme, etc). Other active ingredients include hydrogen peroxide, sodium bicarbonate, arginine, meglumine or zinc salts. Optionally, an anti-microbial is used including but not limited to triclosan, cetylpryidinium chloride, benzalkonium chloride, cetylperidium chloride and cetylmethylammonium bromide, chlorhexidine, chlorine dioxide, dehysoascorbic acid.

A combination of dental health and caries prevention film product containing clove oil or eugenol as an antimicrobial, sodium fluoride as an enamel strengthening fluoride source and arginine as alkaline mouth pH modulator is expressly contemplated. Teeth whitening actives may be employed as an active ingredient. Optionally, the film or sheet adheres to the teeth. The film may comprise agents necessary to adhere the film or sheet to the teeth in the matrix, or such agents may be applied to the film or sheet after processing.

Other embodiments include nutraceutical products (like vitamins, minerals and dietary supplements). Caffeine is a preferred active ingredient.

Film or sheet cosmetics or cosmeceuticals are also contemplated.

Non-cannabinoid botanical ingredients are contemplated. A preferred embodiment is tobacco. Tobacco is typically ground, and preferred embodiments employ tobacco with a moisture content below 12%, more preferably below 10% and most preferably below 8%. Preferably, the tobacco's nicotine content is 2.5% or greater, preferably 3% or greater, more preferably 4% or greater, most preferably 5% or greater. In certain embodiments, the tobacco is pasteurized.

In tobacco embodiments, it is preferable that the final product has a level of tobacco specific nitrosamines below 3 ppm, preferably below 2 ppm, more preferably below 1 ppm, and most preferably below 0.5 ppm.

Active pharmaceutical ingredients are contemplated, whether currently approved, previously approved, or approved in the future.

Non-limitative examples of useful active pharmaceutical ingredients include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulants, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, and combinations thereof.

Examples of active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine. NaV1.8 inhibitors may be used.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film and sheet compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenergic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

Anti-inflammatory agents include steroidal anti-inflammatory drugs, such as cortisone, triamcinalone, prednisone, prednisolone, and the like.

Dental care products are expressly contemplated, including films or sheets with fluoride, local anesthetics, locally acting steroids or antifungals/antibiotics for local infections.

Tooth de-sensitizers may be employed, including inter alia, potassium citrate, potassium chloride, potassium nitrate, strontium chloride, strontium acetate, arginine, calcium carbonate, hydroxyapatite, and calcium sodium phosphosilicate.

Veterinary application and use are expressly contemplated, for livestock and companion animals.

### Stability

Certain embodiments are largely non-hygroscopic, and can be packed using non-oxygen barrier materials with relatively high water vapor permeability, without sticking together. A nitrogen (or other gaseous) blanket may be optionally employed.

In embodiments, the film/sheets are packaged loosely without being clumping together (i.e., not individually packed) in a container with a water vapor permeability of 10⁻⁷ at 22° C. or greater, preferably 10⁻⁸ at 22° C. or greater (cm²*s⁻¹@25° C.).

### Additional Shapes and Configurations

The containers and container components of the invention may be designed or manufactured in myriad shapes and configurations. In embodiments, the container and container components such as the casing may be substantially cylindrical. Other shapes contemplated for the container and container components include, but are not limited to: spherical shapes, tear-drop shapes, pear shapes, crescent shapes, cuboid shapes, prismatic shapes, polygonal shapes, and other shapes suitable for the purposes of the present invention and known in the art.

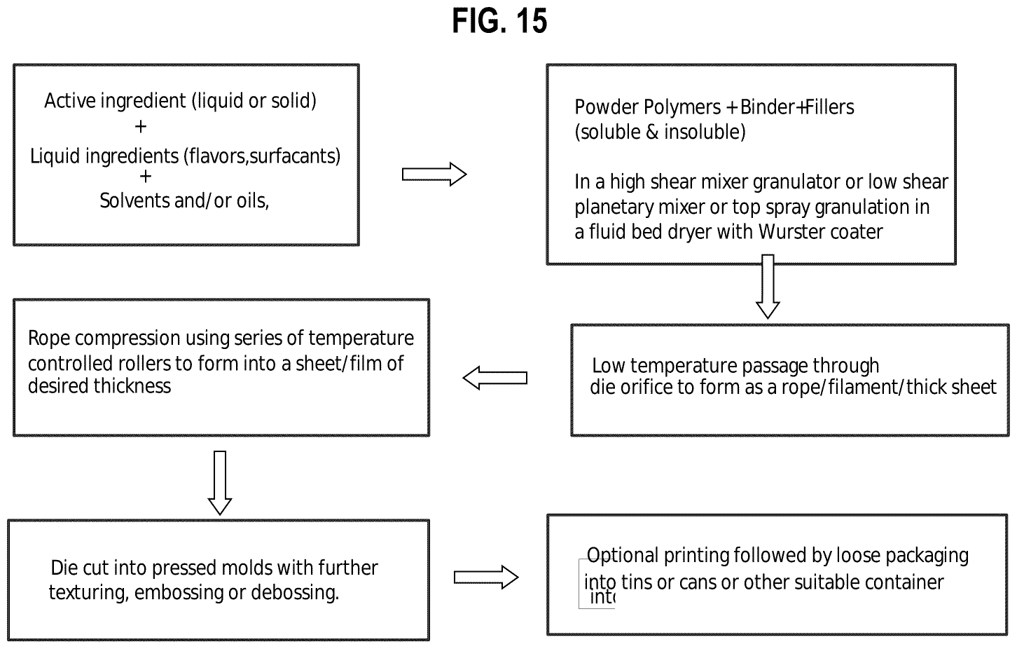

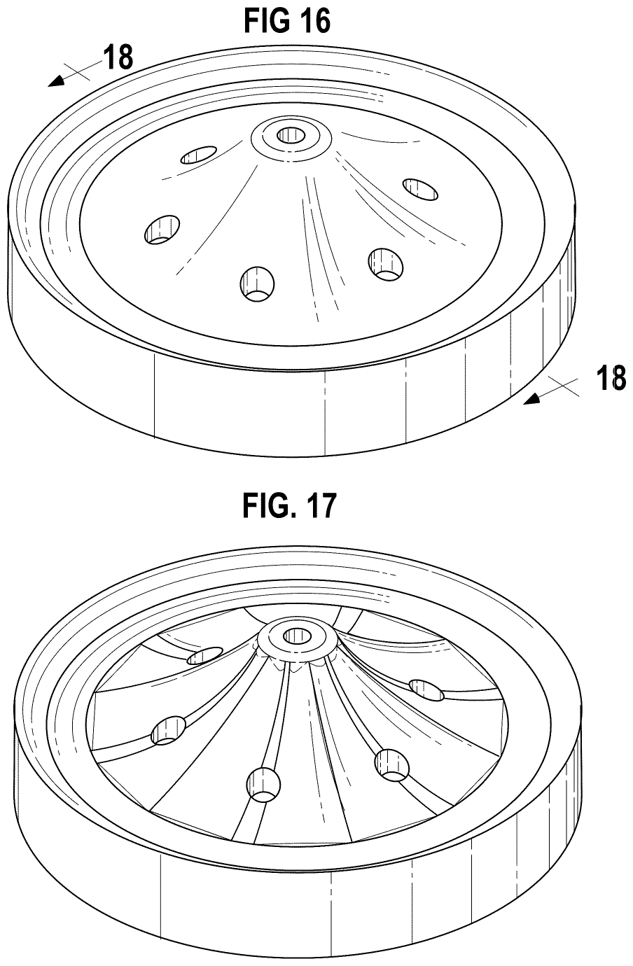

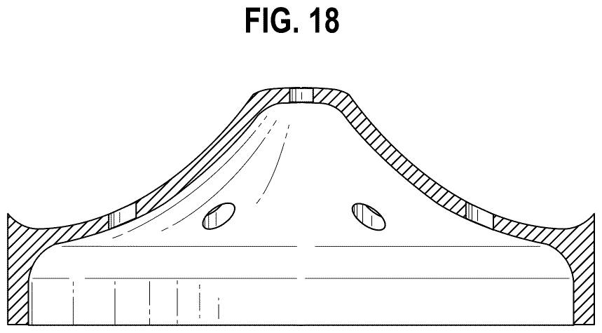

In aspects, the film/sheets have shapes that are not flat. Bent, or curved shapes, such as those of FIGS. 13, 14 and 15 may be particularly suitable for use over the teeth, for suitable applications such as teeth whitening, teeth de-sensitization and other tooth and/or gum issues.

### Film and Sheet Product Configurations

In certain embodiments, of the present invention, the film-sheet product is a multi-layer composition inclusive of one or more films or sheets (i.e., different layers). Multi-layer compositions may comprise two or more layers. Layers may be co-formed, i.e. simultaneously formed, optionally in two different forming vessels, and then joined together when still warm, optionally using rollers to compress the films. Alternatively, layers may be joined after initial manufacture, in which heat, compression and solvents may be used to join the layers together. Water is a preferred solvent. In one embodiment, moisture is applied to one or more layers prior to joining the two layers with compression (typically rollers).

Thickness of the layers may be the same or different. In certain embodiments, one layer is at least 15% thicker than at least one other layer, preferably at least 20% thicker, more preferably at least 30% thicker.

Optionally, one or more layers are textured at the seam of lamination.

Disintegration times of the layers—which may be considered with the two films when apart (i.e. prior to being joined), or also considered when the two films are together—may vary. In certain embodiments, layer disintegrates at least 15% faster than at least one other layer, preferably at least 20% faster, more preferably at least 30% faster.

Compositions of the layers may differ in the multi-layer composition. The layers may have different colors, different flavors, and may comprise different active ingredients (layers without actives may be included).

While typically, the layers will have the same geometry (i.e. same shape) even if thickness optionally differs, in certain embodiments, layers may have differing geometries. Optional functionalities include a mucoadhesive layer, and a backing layer to retard salivary flow.

A three layer film containing a flavor layer laminated between two nicotine containing layer is expressly contemplated. The layers may be laminated by heat, solvent and pressure.

The product may also be supplied as a long tape wound around itself which may be unwound and from which individual pieces may be cut or torn off by the end user.

In still another embodiment, the film or sheet is used to form a pouch, optionally in conjunction with a non-dissolvable material, optionally a fabric material. In preferred embodiments, no non-dissolvable material is used. The pouch may contain a dry blend, a liquid blend, or a dry and liquid blend. The pouch may have one or more compartments.

While much of the disclosure herein refers to films and sheets, the disclosures herein expressly contemplate other shapes, and all disclosures herein referring to films and sheets may also apply to such other non-film/sheet shapes.

In certain embodiments, the length of a film or sheet is no more than 5% the length of the container it is housed within. In other embodiments, the length of the film or sheet is no more than 10% the length of the container it is housed within. In other embodiments, the length of the film or sheet is no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 100%, no more than 105%, no more than 110%, no more than 115%, no more than 120%, no more than 125%, no more than 130%, no more than 140% or no more than 150% the length of the container it is housed within.

In additional embodiments, the length of a film or sheet is about 5% the length of the container it is housed within. In other embodiments, the length of the film or sheet is about 10% the length of the container it is housed within. In other embodiments, the length of the film or sheet is about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 140% or about 150% the length of the container it is housed within.

In yet other embodiments, the length of a film or sheet is at least 5% the length of the container it is housed within. In other embodiments, the length of the film or sheet is at least 10% the length of the container it is housed within. In other embodiments, the length of the film or sheet is at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 140% or at least 150% the length of the container it is housed within.

In embodiments, the container of the present invention includes at least one aperture, at least two apertures, at least three apertures, at least four apertures, at least five apertures, at least six apertures, at least seven apertures, at least eight apertures, at least nine apertures, at least 10 apertures, at least 11 apertures, at least 12 apertures, at least 13 apertures, at least 15 apertures, at least 20 apertures or more.

In yet other embodiments the container of the present invention includes no more than one aperture, no more than two apertures, no more than three apertures, no more than four apertures, no more than five apertures, no more than six apertures, no more than seven apertures, no more than eight apertures, no more than nine apertures, no more than 10 apertures, no more than 11 apertures, no more than 12 apertures, no more than 13 apertures, no more than 15 apertures, or no more than 20 apertures.

In embodiments, the dispenser of the present invention dispenses at least one product per actuation, at least two products per actuation, at least three products per actuation, at least four products per actuation, at least five products per actuation, at least six products per actuation, at least seven products per actuation, at least eight products per actuation, at least nine products per actuation, at least ten products per actuation or more.

In other embodiments, the dispenser of the present invention dispenses no more than one product per actuation, no more than two products per actuation, no more than three products per actuation, no more than four products per actuation, no more than five products per actuation, no more than six products per actuation, no more than seven products per actuation, no more than eight products per actuation, no more than nine products per actuation, or no more than ten products per actuation.

In embodiments, the chamber of the present invention holds at least one product, at least two products, at least three products, at least four products, at least five products, at least six products, at least seven products, at least eight products, at least nine products, at least 10 products, at least 11 products, at least 12 products, at least 13 products, at least 14 products, at least 15 products, at least 20 products, at least 25 products, at least 30 products, at least 35 products, at least 40 products, at least 45 products, at least 50 products, at least 55 products, at least 60 products, at least 65 products, at least 70 products, at least 75 products, at least 80 products, at least 90 products, at least 100 products, at least 110 products, at least 125 products, at least 150 products, at least 200 products, at least 250 products or more.

In other embodiments, the chamber of the present invention holds about one product, about two products, about three products, about four products, about five products, about six products, about seven products, about eight products, about nine products, about 10 products, about 11 products, about 12 products, about 13 products, about 14 products, about 15 products, about 20 products, about 25 products, about 30 products, about 35 products, about 40 products, about 45 products, about 50 products, about 55 products, about 60 products, about 65 products, about 70 products, about 75 products, about 80 products, about 90 products, about 100 products, about 110 products, about 125 products, about 150 products, about 200 products, about 250 products or more.

In embodiments, the chamber of the present invention holds no more than one product, no more than two products, no more than three products, no more than four products, no more than five products, no more than six products, no more than seven products, no more than eight products, no more than nine products, no more than 10 products, no more than 11 products, no more than 12 products, no more than 13 products, no more than 14 products, no more than 15 products, no more than 20 products, no more than 25 products, no more than 30 products, no more than 35 products, no more than 40 products, no more than 45 products, no more than 50 products, no more than 55 products, no more than 60 products, no more than 65 products, no more than 70 products, no more than 75 products, no more than 80 products, no more than 90 products, no more than 100 products, no more than 110 products, no more than 125 products, no more than 150 products, no more than 200 products, no more than 250 products or no more than 500 products.

In certain embodiments, an individual chamber of the present invention includes one protrusion, two protrusions, three protrusions, four protrusions, five protrusions, six protrusions, seven protrusions, eight protrusions, nine protrusions, ten protrusions or more.

In other embodiments, an individual chamber of the present invention includes at least one protrusion, at least two protrusions, at least three protrusions, at least four protrusions, at least five protrusions, at least six protrusions, at least seven protrusions, at least eight protrusions, at least nine protrusions, at least 10 protrusions or more.

In yet other embodiments, an individual chamber of the present invention includes no more than one protrusion, no more than two protrusions, no more than three protrusions, no more than four protrusions, no more than five protrusions, no more than six protrusions, no more than seven protrusions, no more than eight protrusions, no more than nine protrusions, no more than 10 protrusions or no more than 15 protrusions.

In certain embodiments, the height of the chamber containing a film or sheet is no more than 75% of the height of the film or sheet. In other embodiments, the height of the chamber containing a film or sheet is no more than 80% of the height of the film or sheet. In yet other embodiments the height of the chamber containing a film or sheet is no more than 85%, no more than 90%, no more than 95%, no more than 100%, no more than 105%, no more than 110%, no more than 115%, no more than 120%, no more than 125%, no more than 150%, no more than 175%, or no more than 200% of the height of the film or sheet.

In certain embodiments, the height of the chamber containing a film or sheet is about 50% of the height of the film or sheet. In other embodiments, the height of the chamber containing a film or sheet is about 55% of the height of the film or sheet. In yet other embodiments the height of the chamber containing a film or sheet is about 60%, about 65%, about 70%, about 75%, about 80%, about 85%, about 90%, about 95%, about 100%, about 105%, about 110%, about 115%, about 120%, about 125%, about 150%, about 175%, or about 200% of the height of the film or sheet.

In certain embodiments, the height of the chamber containing a film or sheet is at least 75% of the height of the film or sheet. In other embodiments, the height of the chamber containing a film or sheet is at least 80% of the height of the film or sheet. In yet other embodiments the height of the chamber containing a film or sheet is at least 85%, at least 90%, at least 95%, at least 100%, at least 105%, at least 110%, at least 115%, at least 120%, at least 125%, at least 150%, at least 175%, or at least 200% of the height of the film or sheet.

In embodiments, the non-smooth chamber surface area relative to total chamber surface area of the present invention is at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%.

In other embodiments, the non-smooth chamber surface area relative to total chamber surface area of the present invention is about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%.

In yet other embodiments, the non-smooth chamber surface area relative to total chamber surface area of the present invention is no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%, no more than 99.5%.

Further illustration of the present invention is shown in the working examples produced below.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example A

### Efficacy of Orally Soluble Nicotine Films

Smoking is a practice in which a substance is combusted and the resulting smoke is inhaled to be tasted and absorbed into the bloodstream of a person. Most commonly, the substance used is the dried leaves of the tobacco plant, which have been rolled with a small rectangle of paper into a cigarette. Other forms of smoking include the use of a smoking pipe or a bong.

Smoking has negative health effects, because smoke inhalation inherently poses challenges to various physiologic processes such as respiration. Smoking tobacco is among the leading causes of many diseases such as lung cancer, heart attack, COPD, erectile dysfunction, and birth defects. Diseases related to tobacco smoking have been shown to kill approximately half of long-term smokers when compared to average mortality rates faced by non-smokers. Smoking caused over five million deaths a year from 1990 to 2015. Non-smokers account for 600,000 deaths globally due to second-hand smoke.

An average cigarette yields about 2 mg of absorbed nicotine. Nicotine is highly addictive. Because of this, authorities have promoted efforts to curb nicotine addiction (and reduce the craving to smoke). Slow-release forms (e.g., gums and patches) can be less addictive and help in quitting. Controlled levels of nicotine are given to patients through gums, dermal patches, lozenges, inhalers, or nasal sprays to wean them off their dependence. Other methods of nicotine consumption include oral sashets (that are spit out) and chewables/gummies. Each has shortcomings such as, for example, the inability to release nicotine at a steady, controlled rate for an extended period of time.

Recent efforts at smoking cessation include the use of dissolvable films. However, conventional films dissolve quickly and cannot hold much nicotine. In aspects, the films described herein a formulated to hold effective levels of nicotine and dissolve slowly (e.g., 10 minutes or longer) and allow relatively constant release of nicotine (or other agent) through the mucosa as they dissolve.

In this example, a 25 year old male visits a healthcare provider who advises him to quit smoking because of breathing problems and anticipated future complications. Smoking dependence appears partly related to the “high & fast” rise in plasma nicotine concentration achieved by cigarettes. This example illustrates that a nicotine delivery system that provides cigarette-like plasma levels, serves to reduce acute craving, inhibit relapse, and result in higher smoking cessation rates compared with conventional methods.

The patient is provided films containing 0.25% w/w nicotine. The patient dispenses an individual film as needed, when he feels the craving to smoke. The film gradually dissolves and nicotine is released. The nicotine is absorbed through the mucosa in a rate comparable to the rate of nicotine absorption through the lungs via conventional cigarette smoking. The patient is able to replace cigarettes with oral films. Gradually, the patient reduces his use of films thereby reducing his reliance on nicotine.

Further discussion of the confluence of certain factors is appropriate, particularly as relates to embodiments of the invention that have oily ingredients, including nicotine oil and cannabinoid active ingredients, as well as the certain properties that apply to consumer products (as well as other products) in this category.

First, for a consumer product, the absence of individual sachet is highly desirable for ease of access to the product. However, not having an individual sachet for a film product is difficult (hence the dearth of products packaged in multi-film cassettes). Stacking films in a multi-film cassette requires very low film/sheet tackiness; oily films or sheets cannot achieve low levels of tackiness sufficient to permit stacking of films. For example, an 8 mg nicotine oil containing film or sheet that weighs 125 mg is over 6% nicotine oil in the film/sheet matrix. A 12 mg nicotine film or sheet that weighs 125 mg is over 9% nicotine oil in the film/sheet matrix.

Additionally, while the container that holds the oil films or sheets may have a moisture barrier overwrap, at some point, the container overwrap will be removed. Assuming the container is not air-tight (even if air tight, moisture incursion may occur when the container is opened to use the product), ambient moisture incursion becomes an issue. The films or sheets must have sufficient physical stability such that film/sheet shape is not degraded by such exposure to moisture (see discussion of dimensional stability infra).

Because may films/sheets are intended for buccal use, it is important that a film or sheet have level of stiffness to allow it to be easily placed in the check (a more flexible film is harder to place and harder to manipulate in the mouth). In traditional film manufacture and formulation, flexibility of the film is prized as an attribute, so the film can be readily manipulated when it is rolled up after casting and then in subsequent conversion steps to package the film.

Additionally, absorption of ambient moisture by the film/sheet may increased the tackiness of the film/sheet. The film/sheet may adhere to another film or sheet (where the films or sheets are not separated in the container-see container embodiments, infra), or adhere to the surface of the container (see discussions of container surfaces, infra). The film composition should be resistant to moisture uptake, and the composition, in tandem with features of the container, not stick or adhere to the container surfaces during the expected time the container will be exposed to ambient air without barrier protection.

All of these attributes must be pursued in conjunction with the desired attributes of film/sheet performance, i.e. release of active, release of flavor, disintegration time, lack of film residue, desired mucoadhesion, and other desired attributes.

Table 2 below shows the percentage composition of ingredients and excipients for a synthetic nicotine orally soluble film.

TABLE 2
Synthetic Nicotine and Excipients

| | Label | | | | |
|---|---|---|---|---|---|
| | Claim | | Over- | | Melting |
| Ingredients | (mg) | Potency | ages | Percent | pt (C.) |
| Syn Nicotine | 4.00 | 100% | 0 | 2.67% | −79 |
| Excipients | | | | | |
| Klucel ELF | | | | 85.88% | |
| Meglumine | | | | 2.00% | 128-131 |
| Sucralose | | | | 3.00% | 257 |
| Glyceryl Monooleate | | | | 0.05% | 18 |
| Flavor 1 | | | | 3.00% | NA |
| Flavor 2 | | | | 0.25% | NA |
| Ca Silicate | | | | 3.00% | NA |
| Color (FD&C Blue | | | | 0.15% | NA |
| #1) | | | | | |
| TOTAL SOLIDS | | | | 100.00% | |

The ingredients of Example A were melted using staged mixing. Nicotine was deposited or sprayed on to the inactive ingredients with high intensity mixing. The dry feedstock was softened using an extruder at a processing temperature of 90 C. The. The resulting material was run through unheated rollers to control thickness and texturize the resulting sheet.

Individual doses were die cut with a piece weight of 150 mg. Weight variation was tight confirming successful thickness control by dynamic roll molding.

A healthy adult volunteer uses the product in the buccal cavity. Observations included: the stiff film was easy to insert; the relatively low height of the product allowed the product to be used in the buccal cavity below the tooth line; there was an initial nicotine “bite” within 45 seconds of insertion (though the user deemed the “bit” to be mold); the film/sheet took approximately twenty-five minutes to dissolve; the user felt the flavor delivery from the product for the entire dissolution period.

The volunteer believed that the films of Example A provided greater nicotine delivery than an equivalent, 4 mg nicotine pouch.

It was observed that the texture on the film appeared to meaningfully increase surface area.

Twenty of the cut films where plastic in a plastic container, taken from “On!™” nicotine pouch container (which has a rectangular shape). The adult volunteer carried the product in their pocket to determine whether, in real world conditions, product tackiness would be an issue. It was noted in this regard that the interior space was adequate for the film strips to move around while naturally shaken in the pocket. Each dimension of the “On!” container exceeded twice of both the height and width of the film strips.

It was concluded that glycerol monooleate was adequate to retard product tackiness for a formulation containing nicotine oil. A preferred amount is less than 0.2%, or a range of 0.1% to 0.2%, preferably less than 0.1% or a range of 0.01% to 0.1%, more preferably less than 0.075% or a range of 0.01% to 0.075%.

### Example B

TABLE 3
Synthetic Nicotine and Excipients

| Ingredients | Percent |
|---|---|
| Synthetic Nicotine | 1.6% |
| Klucel ELF (HPC) | 81.57% |
| PEO N10 | 0.05% |
| L-Arginine | 3.00% |
| Gelucire | 0.20% |
| MCT oil | 1.00% |
| Mint | 1.50% |
| Aeropearl 300 P | 6.00% |
| Sucralose | 4.00% |
| Magnasweet | 1.00% |
| Blue 1 | 0.08% |
| TOTAL SOLIDS | 100.00% |

The inactive ingredients of Example B were mixed using staged mixing. Nicotine was deposited or sprayed on to the inactive ingredients with high intensity mixing. The dry feedstock was softened using an extruder at a processing temperature of 90 C. The resulting material was run through unheated rollers to control thickness and texturize the resulting sheet.

Individual doses were die cut with a piece weight of 125 mg for a dose of 2 mg.

The volunteer believed that the films of Example A provided greater nicotine delivery than an equivalent, 2 mg nicotine pouch.

Film attributes were judged good in terms of product stickiness and flexibility.

### Example C

TABLE 4

| Ingredients | Percent |
|---|---|
| Synthetic Nicotine | 1.74% |
| Soluplus | 21.29% |
| Klucel ELF (HPC) | 44.99% |
| Corn Syrup Solids | 12.98% |
| PEO N10 | 0.10% |
| L-Arginine | 3.00% |
| Gelucire | 0.20% |
| MCT oil | 1.00% |
| Blood Orange | 3.00% |
| Mint | 0.25% |
| Aeropearl 300 P | 6.00% |
| Sucralose | 4.00% |
| Magnasweet | 0.70% |
| Colorants (yellow 5, 6, Red 50) | 0.75% |
| TOTAL SOLIDS | 100.00% |

The inactive ingredients of Example C were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet. Individual doses were die cut with a piece weight of 125 mg for a dose of 2 mg.

The films of this example were deemed to have improved mouthfeel over the films of Example B, resulting in a less “gummy” feel in the buccal pouch.

### Example D

TABLE 5

| Ingredients | Percent |
|---|---|
| Synthetic Nicotine | 3.2% |
| Soluplus | 8% |
| Klucel ELF | 54.2% |
| Corn Syrup Solids | 15.5% |
| PEO N10 | 0.05% |
| L-Arginine | 3% |
| Gelucire | 0.2% |
| MCT Oil | 1% |
| Blood Orange | 3% |
| Mint | 0.25% |
| Aeropearl 300 P | 6% |
| Sucralose | 4% |
| Magnasweet | 1% |
| Colorants | 0.6% |
| TOTAL SOLIDS | 100% |

The inactive ingredients of Example D were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet. Individual doses were die cut with a piece weight of 125 mg for a dose of 4 mg.

The films of this example had an excellent mouth feel. It was observed that corn syrup solids speeded up disintegration time of the film.

### Example E

TABLE 6

| Ingredients | Percent |
|---|---|
| Synthetic Nicotine Bitartrate | 11.40% |
| Klucel ELF | 40.45% |
| Erythritol | 29.10% |
| L-Arginine | 3% |
| MCT oil | 0.50% |
| Blood Orange | 3.00% |
| Mint | 0.25% |
| PEO | 1.00% |
| Aerosil 300 P | 6.00% |
| Sucralose | 4.00% |
| Magnasweet | 0.70% |
| Colorants Yellow & Red | 0.6% |
| TOTAL SOLIDS | 100% |

The inactive ingredients of Example E were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet.

Individual doses were die cut with a piece weight of 100 mg for a dose of 4 mg.

The formulation was adjudged to be too soft.

### Example F

TABLE 7

| Ingredients | Percent |
|---|---|
| Synthetic nicotine | 8.00% |
| Klucel ELF | 74.85% |
| L-Arginine | 2.00% |
| MCT oil | 0.50% |
| Blood Orange | 3.00% |
| Mint | 0.25% |
| PEO | 0.10% |
| Ca Silicate | 6.00% |
| Sucralose | 4.00% |
| Magnasweet | 0.70% |
| Colorants - Yellow 5, 6, Red 40 | 0.55% |
| TOTAL SOLIDS | 100% |

The inactive ingredients of Example F were mixed using staged mixing. Nicotine was deposited or sprayed on to the inactive ingredients with high intensity mixing. The dry feedstock was softened in an extruder at a processing temperature of 90 C. The resulting semi-molten material was run through unheated rollers to control thickness and texturize the resulting sheet. Individual doses were die cut with a piece weight of 50 mg for a dose of 4 mg.

The film was adjudged to be too soft.

### Example G

TABLE 8

| Ingredients | Percent |
|---|---|
| Syn Nicotine | 2.67% |
| Klucel ELF | 83.05% |
| L-Arginine | 2% |
| MCT oil | 0.5% |
| Menthol | 0.5% |
| Clove oil | 1.5% |
| Aerosil r972 | 4% |
| Ca Silicate | 2% |
| Sucralose | 3% |
| Magnasweet | 0.7% |
| Blue 1 | .08% |
| TOTAL SOLIDS | 100% |

The inactive ingredients of Example G were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet. The pieces were cut to a weight of 150 mg. The Formulation took approximately seventeen minutes to dissolve in the buccal cheek pouch of an adult user. Film properties were good.

### Example H

TABLE 9

| Ingredients | Percent |
|---|---|
| Syn Nicotine | 5.33% |
| Klucel ELF | 80.39% |
| L-Arginine | 2% |
| MCT oil | 0.5% |
| Menthol | 0.5% |
| Clove oil | 0.5% |
| Aerosil R972 | 4% |
| Ca Silicate | 2% |
| Sucralose | 4% |
| Magnasweet | 0.7% |
| Blue 1 | .08% |
| TOTAL SOLIDS | 100% |

The inactive ingredients of Example H were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet.

The pieces were cut to a weight of 150 mg, for a target dose of 8 mg per piece. The Formulation took approximately eighteen minutes to dissolve in the buccal cheek pouch of an adult user. Film properties were good.

### Example I

TABLE 10

| Ingredients | Percent |
|---|---|
| Delta 8 THC | 3.33% |
| Klucel ELF | 87.47% |
| PEO N10 | 6% |
| Caprol PGE-860 | 0.25% |
| GMO (Peceol) | 0.25% |
| Meglumine | 0.5% |
| Sucralose | 2% |
| Ca Silicate | 3% |
| Aerosil R972 | 2% |
| Magnasweet | 0.1% |
| Magna CBD | 0.05% |
| Grapefruit Flavor | 4% |
| TOTAL SOLIDS | 100% |

The inactive ingredients of Example 1 were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet.

Pieces were cut to 150 mg, for an intended dose of 5 mg.

The formulation comprised a combination of surfactants—Caprol and Peceol. This is a combination of a high HLB surfactant (10-12) (Caprol PGE-860) with a low HLB surfactant (0-1) (Peceol). In this formulation they are used in a 1-1 ratio; a ratio of 1-3 is also contemplated.

### Example J

TABLE 11

| Ingredients | Percent |
|---|---|
| Syn Nicotine | 3.20% |
| Klucel ELF | 63.33% |
| Corn Syrup Solids | 15.50% |
| PEO N10 | 0.07% |
| L-Arginine | 3.00% |
| Blood Orange | 3.00% |
| Mint | 0.25% |
| Aeropearl 300 P | 6.00% |
| Sucralose | 4.00% |
| Magnasweet | 1.00% |
| Yellow5 | 0.40% |
| Yellow6 | 0.15% |
| Red40 | 0.10% |
| TOTAL SOLIDS | 100.00% |

The inactive ingredients of Example J were mixed using staged mixing. Nicotine was deposited or sprayed on to the inactive ingredients with high intensity mixing. The dry feedstock was softened in a heated barrel at a processing temperature of 90 C. The resulting semi-molten material was run through unheated rollers to control thickness and texturize the resulting sheet.

Pieces were cut to 150 mg, for an intended dose of 4 mg.

The formulation did not contain surfactants.

### Example K

### Single Dose Dispensing of Orally Soluble CBD Product

A subject in clinical trial for an orally soluble CBD film product dispenser utilized the dispensing device of the present invention. To operate the device, the subject rotated an outer cylindrical case of a cylindrically shaped dispenser until an open chamber within the device aligned with an open aperture on the cylindrical case.

Once the chamber was aligned with an open aperture, an oral film product containing 25% CBD (cannabidiol) was dispensed by gravity into the subject's hand.

After receiving the dose, the subject noted that the chamber aligned with the aperture was empty. To retrieve another dose, the subject would have to rotate the cylindrical case until the next successive chamber aligned with the open aperture of the dispensing device.

It is expressly contemplated that embodiments of the present invention may be used with dosage forms other than films or sheets; including without limitation, tablets, capsules, gel-caps, lozenges, nicotine pouches, non-nicotine pouches with other actives, and other solid dosage forms.

Accordingly, embodiments of the invention include a dispenser for releasing pieces of oral film. The dispenser can include (a) a central hub, (b) a plurality of spokes that protrude from the central hub to a periphery to form individual chambers, and (c) an outer case with an aperture. In aspects, the central hub is configured to rotate around an axis so that a chamber aligns with an aperture to release a piece of the oral film. In aspects, each spoke is wider at its periphery (e.g., arrow-shaped) to create positional stability for a single piece of oral film. In aspects, each piece of the oral film includes an active agent (e.g., nicotine).

In embodiments, the dispenser includes a push-button actuator that rotates a first chamber from the aperture to a second chamber.

Embodiments also include a dispenser that is shaped like a puck. The puck dispenser can include a central chamber portion that is elevated (i.e., to form a cone shape).

Embodiments also include an oral dissolvable film. The film can include: (a) about 3% or more nicotine oil, (b) about 1-5% w/w of an oil absorbent agent and (c) a basic amino acid as a pH modifier. In aspects, the film has a pH of 7 or higher, and the oil absorbent agent is silicone dioxide, calcium silicate, magnesium stearate, magnesium silicate, soluble fibers and/or a gum.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter. Further, the use of the terms “include,” “includes” and “including” means include, includes and or including as well as include, includes and including, but not limited to.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

Although embodiments of the current disclosure have been described comprehensively in considerable detail to cover the possible aspects, those skilled in the art would recognize that other versions of the disclosure are also possible.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

What is claimed is:

1. A dispenser for releasing pieces of oral film, the dispenser comprised of:
a) a central hub,
b) a plurality of spokes that protrude from the central hub to a periphery to form individual chambers, and
c) an outer case with an aperture,
wherein the central hub is configured to rotate around an axis so that a chamber aligns with an aperture to release a piece of the oral film,
wherein each spoke of the plurality of spokes, and
wherein each piece of the oral film is comprised of a dose of an active agent.

2. The dispenser of claim 1, further comprising a push-button actuator that rotates a first chamber from the aperture to a second chamber.

3. The dispenser of claim 1, wherein at least one surface of the chambers is comprised of a textured surface.

4. The dispenser of claim 1, wherein each piece of the oral film has a length by width aspect ratio of about 1.5 to 1 or more.

5. The dispenser of claim 1, wherein the active agent is a cannabinoid, nicotine or nicotine oil.

6. The dispenser of claim 5, wherein the oral film comprises 3% w/w or more of nicotine or nicotine oil.

7. The dispenser of claim 1, wherein the oral films comprise Soluplus and HPC, wherein the ratio of Soluplus to HPC is between about 1-9 to 1-3.

8. The dispenser of claim 1, wherein the oral films comprise Soluplus and HPC, wherein the ratio of Soluplus to HPC is between about 1-8 to 1-4.

9. The dispenser of claim 1, wherein the oral films have dimensional stability, wherein dimensional stability is determined by exposing the films or sheets to a temperature of about 30° C. and about 65% relative humidity (RH) for 24 hours and determining that the films or sheets bend less than 1.5% in either of the length or height direction.

10. The dispenser of claim 5, wherein the oral films further comprise about 8% w/w to 15% w/w of corn syrup solids.

11. The dispenser of claim 5, wherein the oral films further about 5% to 15% w/w sodium caprate.

12. The dispenser of claim 5, wherein the oral films further comprise about 0.5% w/w to 2.5% w/w medium-chain triglyceride (MCT).

13. A puck dispenser for releasing pieces of oral film, the dispenser comprised of a central chamber,
wherein the central chamber is elevated to form a cone shape to allow a user to remove pieces of oral film from the dispenser.

14. The dispenser of claim 13, wherein the chamber surface area is comprised of a material with a surface energy of 30 dynes/cm or less.

15. The dispenser of claim 13, wherein the oral films are comprised of about 3% w/w or more of nicotine oil or a cannabinoid.

16. The dispenser of claim 13, wherein the oral films are dusted to reduce product tack.

17. An oral dissolvable film comprised of:
a) about 3% or more nicotine oil,
b) about 1-5% w/w of an oil absorbent agent; and
c) a basic amino acid as a pH modifier,
wherein the film has a pH of 7 or higher, and
wherein the oil absorbent agent is selected from silicone dioxide, calcium silicate, magnesium stearate, magnesium silicate, soluble fibers and gums.

18. The oral dissolvable film of claim 17, wherein the film further comprises about 8-15% w/w corn syrup solids and about 0.7 to 1.25% PEO as a plasticizer.

19. The oral dissolvable film of claim 17, wherein the film or sheet has dimensional stability, wherein dimensional stability is determined by exposing the films or sheets to a temperature of about 30° C. and about 65% relative humidity (RH) for 24 hours and determining that the films or sheets bend less than 3% in either of the length or height direction (where said films/sheets are not in barrier packaging).

20. The oral dissolvable film of claim 17, wherein film further comprises Soluplus and HPC, wherein the ratio of Soluplus to HPC is between about 1:8 to 1:4.
